(19) United States
(12) Patent Application Publication
(10) Pub. No.: US 2023/0233770 A1
(43) Pub. Date: Jul. 27, 2023

(54) DRUG DELIVERY DEVICE SAFETY SYSTEM

(71) Applicant: Regeneron Pharmaceuticals, Inc., Tarrytown, NY (US)

(72) Inventors: Trevor LANGLEY, Rensselaer, NY (US); Bryan GRYGUS, Clifton Park, NY (US); Daniel HALBIG, Ballston Lake, NY (US); Paige WAECHTER, Dorchester, MA (US); Andrew DUMONT, Rensselaer, NY (US); Parker VALDEZ, Mountain View, CA (US); Sindhuja KUCHIBHATLA, Albany, NY (US)

(73) Assignee: Regeneron Pharmaceuticals, Inc., Tarrytown, NY (US)

(21) Appl. No.: 18/159,445

(22) Filed: Jan. 25, 2023

Related U.S. Application Data
(60) Provisional application No. 63/302,989, filed on Jan. 25, 2022.
(60) Provisional application No. 63/305,763, filed on Feb. 2, 2022.
(60) Provisional application No. 63/480,904, filed on Jan. 20, 2023.

(51) Int. Cl. A61M 5/31; A61M 5/315; A61M 5/32

(52) U.S. Cl. CPC A61M 5/3137; A61M 5/31505; A61M 5/3202

(57) ABSTRACT

A drug delivery device is disclosed, wherein the device comprises a housing; a product container arranged in the housing; a plunger rod partially disposed inside the product container; and a thumb pad configured to receive a portion of the plunger rod, wherein the thumb pad and the plunger rod are configured to transition from a first state to a second state, wherein at the first state, the plunger rod is prevented from moving in a proximal direction, and at the second state, the plunger rod moves freely in a proximal direction.

### CROSS-REFERENCE TO RELATED APPLICATIONS

This application claims the benefit of priority from U.S. Provisional Patent Application Nos. 63/302,989, filed Jan. 25, 2022; 63/305,763, filed Feb. 2, 2022; and 63/480,904, filed Jan. 20, 2023, all of which are incorporated by reference herein.

### TECHNICAL FIELD

This disclosure is directed to a drug delivery device (e.g., a prefilled syringe) safety system and uses thereof.

### INTRODUCTION

Drug delivery devices (e.g., prefilled syringes, auto-injectors, or other suitable drug delivery devices) are routinely used to deliver fluid drug substances. Various safety concerns may arise before, during, and after the use of a drug delivery device. To address such concerns, conventional safety devices have been developed. Conventional safety devices may include an active safety mechanism. In such devices, the user is required to actively perform a specific action to trigger a safety mechanism. It may be difficult for a user to properly activate a safety mechanism, while at the same time, properly utilizing the drug delivery device for injecting a drug substance into themselves or another person. In contrast to these active safety devices, passive safety devices have been developed, where there is no need for a user to perform any additional and/or separate action to trigger a safety mechanism during and/or after use of a drug delivery device.

In passive safety systems, there may still be concerns for user safety and proper usage of the drug delivery device. For example, there may be premature expulsion of the fluid, accidental needle stick pre-injection and/or post-injection, and/or premature lockout prior to end of dose. Passive safety mechanisms should be configured for proper and safe usage before, during, and after injection.

### SUMMARY OF THE DISCLOSURE

The present disclosure describes a drug delivery device, the device comprising a housing; a product container arranged in the housing; a plunger rod partially disposed inside the product container; and a thumb pad configured to receive a portion of the plunger rod, wherein the thumb pad and the plunger rod are configured to transition from a first state to a second state, wherein at the first state, the plunger rod is prevented from moving in a proximal direction and at the second state, the plunger rod moves freely in a proximal direction.

Various embodiments of the device may include one or more of the following aspects. The thumb pad may comprise an extension projecting distally from the thumb pad. The housing may comprise a shoulder, wherein in the first state, the shoulder projects radially inwards and prevents movement of the product container. In the second state, the extension of the thumb pad may be configured to deflect the shoulder radially outwards to allow the product container to move in a proximal direction. The product container and the needle attached to a distal end of the product container may retract into the housing in the proximal direction so that the needle is located proximal of a distal end of the housing. A distance the product container retracts proximally into the housing in the second state may be substantially equal to a length of the needle extending distally from the housing in the first state. The proximal end portion of the plunger rod may include a flange and the thumb pad may include a recess configured to receive the flange. The plunger rod may abut an interior distal surface of the thumb pad in the second state. An audible feedback may be produced as a portion of the plunger rod abuts an interior distal surface of the thumb pad in the second state. The housing may include a surface configured to deflect a portion of the thumb pad in an outward direction to release the proximal portion of the plunger rod when transitioning to the second state. The housing may include a cutout configured to engage a portion of the thumb pad to couple the thumb pad to the housing in the second state. The plunger rod may include a first portion and a second portion, the first portion having a diameter greater than a diameter of the second portion, such that in a first state, the first portion is proximal to the second portion and in a second state, the second portion collapses into the first portion. The device may include a rotator configured to sit proximal to a proximal end of the product container in a first state and configured to surround the proximal end of the product container in a second state. The plunger rod may include a first portion and a second portion, a distal end of the second portion being coupled to a proximal end of the first portion, and the housing may include a proximal extension, wherein in a first state, the proximal extension abuts the distal end of the first portion, preventing the product container from moving proximally away from the housing. In a second state, the proximal extension may deflect away from the distal end of the first portion, allowing the product container to move proximally away from housing and into the thumb pad. A proximal end of the housing may include a rear cap configured to surround a proximal end of the product container.

The present disclosure also describes a drug delivery device comprising a housing; a product container disposed in the housing, and a needle extending from a distal end of the product container; a plunger rod for dispensing a product from the product container, the plunger rod including a first indent at a distal portion of the plunger rod and a second indent at a proximal portion of the plunger rod; a blocking component at a proximal end of the housing, wherein the plunger rod is slidably received within the blocking component, and wherein the blocking component includes a deflectable arm; and a needle cover at least partially disposed in the housing.

Various embodiments of the device may include one or more of the following aspects. The device may be configured to transition from a first state in which a distal end of the needle cover abuts the deflectable arm and the deflectable arm is aligned with the first indent, a second state in which the deflectable arm is deflected radially outward, and a third state in which the deflectable arm is aligned with the second indent, and the distal end of the needle cover is located proximal to the deflectable arm. The second indent may have a smaller diameter than the first indent. The housing may include a ledge for abutting a portion of the needle cover to prevent proximal movement of the needle cover into the housing. In the first state, the needle cover may extend distally of the housing to cover the needle. In the second state, the proximal end of the needle cover may abut a distal portion of the blocking component. In the first state, the deflectable arm may restrict movement of the plunger rod, and wherein in the second state, the deflected deflectable arm may permit movement of the plunger rod. The device may include an internal rod configured to be secured within the plunger rod. The internal rod may include a distal block configured to abut a portion of the plunger rod, preventing the plunger rod from moving in a distal direction. A distal end of the plunger rod include deflectable arms configured to abut against a portion of the internal rod, preventing the plunger rod from moving in a distal direction. The plunger rod may include a flexible section, such that in a first state, the distal block prevents the flexible section from compressing and in a second state, the distal block may move away from the flexible section allowing the flexible section to compress. The housing may include a finger flange and a button mechanism, wherein the button mechanism is configured to abut a portion of the plunger rod in a first state and deflect away from the plunger rod in a second state.

The present disclosure also describes a drug delivery device comprising a housing; a plunger rod; a needle cover disposed at least partially within the housing; and a rotating component having a track extending along an outer surface of the rotating component, wherein a portion of the plunger rod extends through a central region of the rotating component, wherein the rotating component is located radially inward of a proximal portion of the needle cover, and wherein a projection of the needle cover engages the track of the rotating component and is configured to move along the track of the rotating component as the device transitions from a first state, to a second state, and to a third state.

Various embodiments of the device may include one or more of the following aspects. The device may further comprise a product container for containing a liquid drug product; and a stopper disposed within the product container, wherein distal movement of the plunger rod distally moves the stopper, and wherein, in the first state, the projection engages a first end of the track, and distal movement of the plunger rod is restricted. In the second state, the projection may engage a middle portion of the track so that distal movement of the plunger rod is permitted, and the needle cover may be positioned further proximally within the housing relative to a position of the needle cover in the first state. In the third state, the projection may engage a second end of the track, and the needle cover may be located further distally within the housing relative to a position of the needle cover in the second state to extend from a distal end of the housing. In the third state, the needle cover may be located further distally within the housing relative to the position of the needle cover in the first state, wherein the needle cover extends from the distal end of the housing in the first state and in the third state.

The present disclosure also describes a drug delivery device comprising a housing comprising a deflectable tab; a syringe arranged in the housing; a plunger rod including a stopper for dispensing a product contained in the syringe; a needle cover at least partially disposed in the housing, the needle cover including a notch; and a ring comprising a ledge, the ring positioned around a proximal end of the syringe, wherein the device is configured to transition between a first state, a second state, and a third state, wherein in the second state, the needle cover is located further proximally within the housing compared to the first state and the third state, the proximal end of the needle cover is configured to push the deflectable tab of the housing outwards, and the notch of the needle cover engages with the ledge of the ring.

Various embodiments of the device may include one or more of the following aspects. In the third state, the needle cover and ring may be located distal to the deflectable tab, and the deflectable tab may restrict movement of the needle cover in the proximal direction. In the first state, the needle cover may be located distal to the ring.

The present disclosure also describes a drug delivery device comprising a housing, a product container arranged in the housing, and a plunger rod partially disposed inside the product container, the plunger rod including a first portion and a second portion, wherein the first portion is configured to receive the second portion, wherein the first portion and the second portion of the plunger rod are configured to transition from a first state to a second state, wherein at the first state, the second portion of the plunger rod is prevented from moving in a proximal direction and at the second state, the second portion of the plunger rod moves freely in a proximal direction.

Various embodiments of the device may include one or more of the following aspects. The portion may include a thumb pad. The thumb pad may include a recess configured to receive the second portion of the plunger rod. In the second state, the second portion of the plunger rod may move in a proximal direction into the first portion of the plunger rod. The plunger rod may include a flexible portion. The second portion of the plunger rod may be removably coupled to the first portion of the plunger rod.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure. Where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIGS. 1A-1B depict an exemplary safety device and components thereof, according to embodiments of the present disclosure.

FIGS. 2A-2F depict additional aspects and embodiments of the exemplary safety device of FIGS. 1A-1B.

FIGS. 3A-3B depict an exemplary safety device and components thereof, according to embodiments of the present disclosure.

FIGS. 4A-4D depict additional aspects and embodiments of the exemplary safety device of FIGS. 3A-3B.

FIGS. 5A-5D depict additional aspects and embodiments of the exemplary safety device of FIGS. 3A-3B.

FIGS. 6A-6B depict an exemplary safety device and components thereof, according to embodiments of the present disclosure.

FIGS. 7A-7F depict additional aspects and embodiments of the exemplary safety device of FIGS. 6A-6B.

FIG. 8A-8B depict an exemplary safety device and components thereof, according to embodiments of the present disclosure.

FIGS. 9A-9D depict additional aspects and embodiments of the exemplary safety device of FIGS. 8A-8B.

FIGS. 10A-10B depict additional aspects and embodiments of the exemplary safety device of FIGS. 8A-8B.

FIG. 11 depicts an exemplary safety device and components thereof, according to embodiments of the present disclosure.

FIGS. 12A-12C depict additional aspects and embodiments of the exemplary safety device of FIG. 11.

FIGS. 13A-13B depict additional aspects and embodiments of the exemplary safety device of FIG. 11.

FIGS. 14A-14C depict an exemplary safety device and components thereof, according to embodiments of the present disclosure.

FIGS. 15A-15D depict additional aspects and embodiments of the exemplary safety device of FIGS. 14A-14C.

FIGS. 16A-16B depict additional aspects and embodiments of the exemplary safety device of FIGS. 14A-14C.

FIGS. 17A-17G depict an exemplary safety device and components thereof, according to embodiments of the present disclosure.

FIGS. 18A-18B depict an exemplary safety device and components thereof, according to embodiments of the present disclosure.

FIGS. 19A-19B depict additional aspects and embodiments of the exemplary safety device of FIGS. 18A-18B.

FIGS. 20A-20C depict additional aspects and embodiments of the exemplary safety device of FIGS. 18A-18B.

FIGS. 21A-21C depict additional aspects and embodiments of the exemplary safety device of FIGS. 18A-18B.

FIGS. 22A-22B depict additional aspects and components of exemplary safety devices, according to embodiments of the present disclosure.

FIGS. 23A-23B depict an exemplary safety device and components thereof, according to embodiments of the present disclosure.

FIGS. 24A-24C depict an exemplary safety device and components thereof, according to embodiments of the present disclosure.

FIG. 25 depicts an exemplary safety device and components thereof, according to embodiments of the present disclosure.

FIGS. 26A-26B depict an exemplary safety device and components thereof, according to embodiments of the present disclosure.

FIGS. 27A-27B depict an exemplary safety device and components thereof, according to embodiments of the present disclosure.

FIGS. 28A-28B depict an exemplary safety device and components thereof, according to embodiments of the present disclosure.

FIGS. 29A-29B depict an exemplary safety device and components thereof, according to embodiments of the present disclosure.

FIGS. 30A-30D depict an exemplary safety device and components thereof, according to embodiments of the present disclosure.

FIG. 31 depicts an exemplary safety device and components thereof, according to embodiments of the present disclosure.

FIGS. 32A-32F depict an exemplary safety device and components thereof, according to embodiments of the present disclosure.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “or” is inclusive and is intended to mean that a process, method, article, or apparatus that comprises a list of elements may include a combination of or all of the elements. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

The term “distal end” or any variation thereof, refers to the portion of the device that is the drug delivery end. Conversely, the term “proximal end” or any variation thereof, refers to the portion of the device that is the actuation end. Further, as used herein, the terms “about,” “substantially,” and “approximately” generally mean+/−10% of the indicated value.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts.

Various safety concerns may arise with the use of a drug delivery device. Safety issues and concerns may occur prior to, during, and/or after use, i.e., injection of a product, e.g., a liquid medicament, from the drug delivery device. For example, premature expulsion of the product may occur if components of the device are not secure and/or locked into a proper pre-injection position. Exposure of the needle may cause accidental needle stick during injection if the device is accidentally removed from the injection site, and/or after injection when the device is removed from the injection site and disposed of. Exposure of the needle may also adversely affect users with needle-phobia and/or needle-related anxiety. In addition, there may be concerns of the user inserting and/or injecting the needle at an incorrect depth into the injection site.

To address such concerns, conventional safety devices have been developed. Conventional safety devices may include active safety mechanisms. Active safety mechanisms refer to mechanisms where the user is required to actively perform a specific action to trigger the safety mechanism. It may be difficult for lay users, i.e., users without medical training, to properly activate an active safety mechanism, while simultaneously properly injecting a medicament into themselves or another person. Further, active safety mechanisms may not have any effect if the user fails to initiate the safety mechanism. Passive safety mechanisms have been developed to address such issues. Devices including passive safety mechanisms do not require the user to perform any additional and/or separate action to trigger a safety mechanism, prior to, during, and/or after use of the device. However, other concerns may occur with the use of a passive drug delivery device. For example, if a user accidentally removes the device from the injection site, a passive safety mechanism may automatically activate prior to completion of the dose. In such case, the user may not be able to complete the dose and would have to obtain another device.

Embodiments of the present disclosure relate to a drug delivery device, and in particular, a device for passively activating a safety mechanism, during, and/or after injection of a medicament from a product container, i.e., syringe.

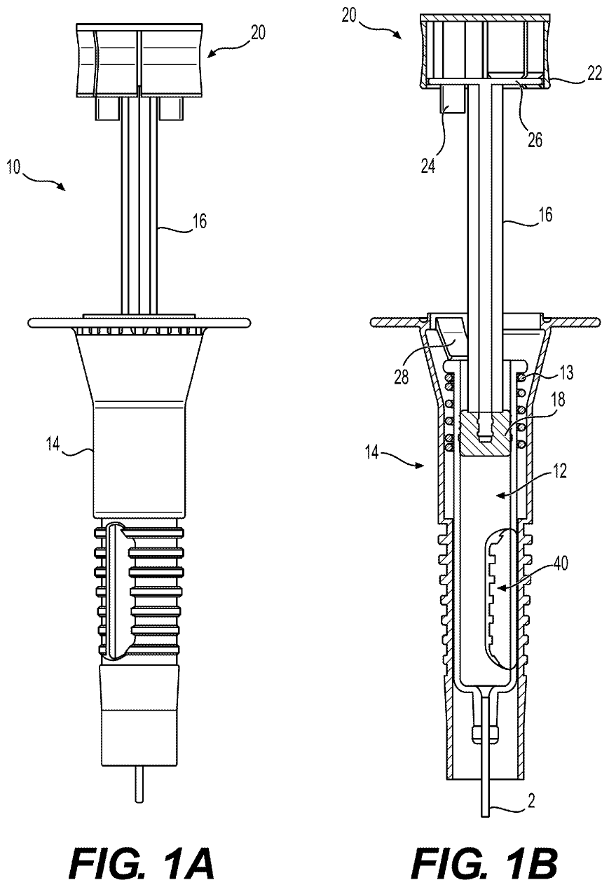

FIG. 1A shows an exterior view of a device 10. Device 10 may be designed to hold any commercially known product container 12 (e.g., a syringe or other suitable drug delivery device). Device 10 may include a housing 14, plunger rod 16, and thumb pad 20. FIG. 1B shows an interior view of device 10, wherein housing 14 may be configured to house container 12. In the embodiment shown in FIG. 1B, container 12 may be a syringe, e.g., a prefilled syringe. Housing 14 may include an inward projecting shoulder 28. As shown in FIG. 1B, shoulder 28 may be located in a proximal portion of housing 14 and may abut a proximal end of container 12 when housed in housing 14. Plunger rod 16 may include a stopper 18 for expelling product from container 12 and a flange 26 for coupling to thumb pad 20. Referring to FIG. 1B, stopper 18 may be positioned at the distal end of plunger rod 16, and flange 26 may be positioned at the proximal end of plunger rod 16.

As will be described in detail below, plunger rod 16 may abut and/or releasably couple to a portion of a syringe, e.g., a piston of the syringe, in housing 14. In some embodiments, plunger rod 16, thumb pad 20, and the syringe, may be locked together during injection, transmitting the user's force on plunger rod 16 to a piston of the syringe. After injection is complete, plunger rod 16 may decouple from the syringe. After plunger rod 16 decouples from the syringe, thumb pad 20 may couple to housing 14, preventing thumb pad 20, plunger rod 16, and/or the syringe, from moving in a proximal direction. As thumb pad 20 moves in a distal direction to housing 14, a spring 13 may move the syringe in a proximal direction into housing 14.

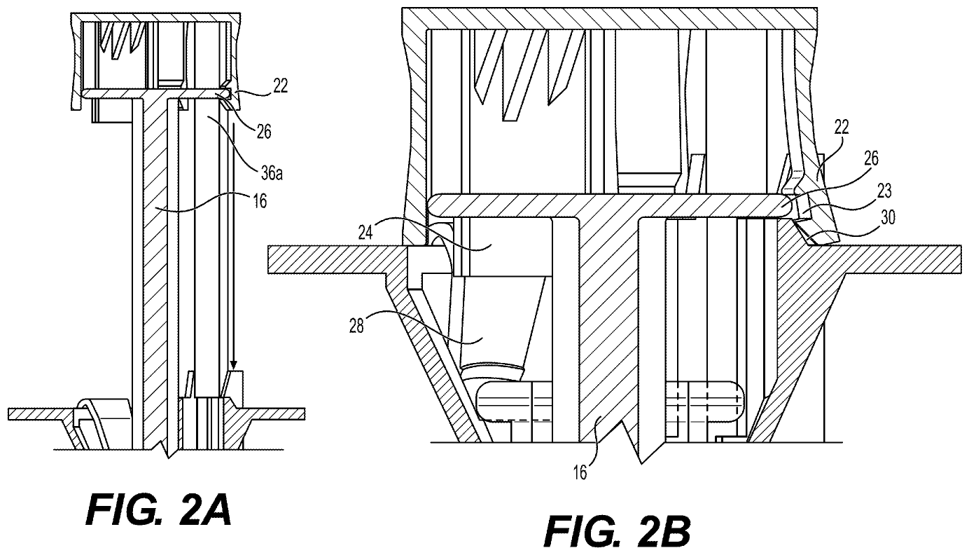

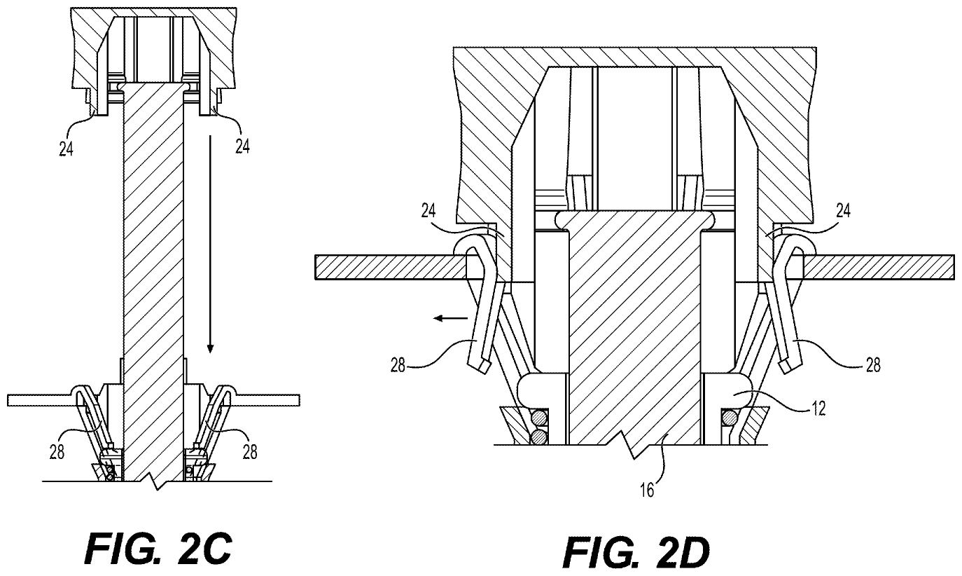

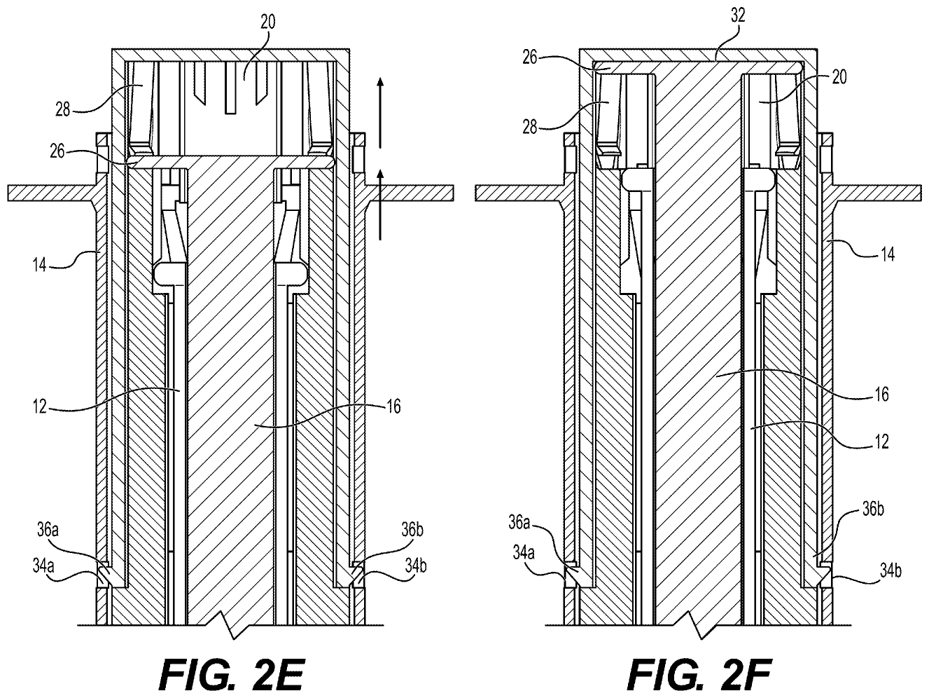

Various components may be utilized to move plunger rod 16 and/or syringe and then lock thumb pad 20, plunger rod 16, and/or the syringe into housing 14. In some embodiments, thumb pad 20 may include a deflectable tab 22 and distal extension 24. As shown in FIG. 2B, deflectable tab 22 may include a recess 23. Recess 23 may be releasably coupled to flange 26 of plunger rod 16. In some embodiments, deflectable tab 22 and recess 23 may be formed continuously or around a majority of an inner portion of thumb pad 20. In other words, deflectable tab 22 and recess 23 may be formed in thumb pad 20 to surround flange 26. In other embodiments, thumb pad 20 may include more than one deflectable tab 22 and recess 23. For example, thumb pad 20 may include two deflectable tabs, three deflectable tabs, or four deflectable tabs, each with a recess 23. In configurations with more than one deflectable tab 22 and recess 23, the tabs may be equally spaced apart from one another. For example, if the thumb pad 20 includes four deflectable tabs, each of the tabs may be about 90 degrees from one another. In addition, each deflectable tab may include a recess 23 for releasably coupling to a portion of flange 26.

In some embodiments, thumb pad 20 may include more than one distal extension 24. For example, thumb pad 20 may include two, three, four, or more distal extensions 24. In configurations with more than one distal extension 24, the distal extensions may be equally spaced apart from one another. For example, if thumb pad 20 includes four distal extensions 24, each of the distal extensions may be about 90 degrees from one another. In some embodiments, a single distal extension may extend continuously or at least partially around thumb pad 20.

Prior to injection, plunger rod 16 may be fully extended from a proximal end of housing 14 (FIG. 1B). In such a configuration, container 12 (e.g., a filled syringe) and plunger rod 16 may not be removed from device 10. Various components may prevent removal of plunger rod 16 from device 10. For example, arm 36a of thumb pad 20 may lock into housing 14 to prevent the removal of plunger rod 16. Referring to FIGS. 2A-2B, to initiate injection of the product from container 12, a user may depress thumb pad 20 to move thumb pad 20 and plunger rod 16 in a distal direction towards housing 14. After the product is fully expelled from container 12 at the end of the injection dose, thumb pad 20 may abut the proximal end of housing 14 (FIG. 2B). The proximal end of housing 14 may include a collar 30 aligned with deflectable tab 22. As shown in FIG. 2B, collar 30 may have a beveled surface configured to abut and flex deflectable tab 22 in an outwards direction away from flange 26 as the injection dose is completed. Deflectable tab 22 may also include a beveled surface configured to abut the beveled surface of collar 30 to facilitate deflection of deflectable tab 22. Thumb pad 20, tab 22, and recess 23, may decouple from flange 26 and release plunger rod 16. FIG. 2A also depicts an arm 36a of thumb pad 20, which will be described in more detail below in reference to the lockout feature of device 10.

Also shown in FIG. 2B, once thumb pad 20 abuts housing 14, at the end of the injection dose, distal extension 24 of thumb pad may contact and deflect shoulder 28 of housing 14 in an outwards direction away from plunger rod 16. FIG. 2C depicts distal extensions 24 and shoulders 28 before they are moved into contact with one another, and FIG. 2D depicts distal extensions 24 of thumb pad 20 as they begin to contact shoulders 28. Once shoulder 28 is deflected in an outwards direction away from plunger rod 16 and the proximal end of container 12, container 12 may be released, allowing for movement of container 12 in a proximal direction. In some examples, a spring 13, e.g., a spring shroud, may abut a portion of container 12 in housing 14, in order to push container 12 in a proximal direction once container 12 is released from shoulder 28. For example, an at least partially compressed spring 13 may contact a distal surface of a flange of container 12, and when shoulder 28 is deflected outwards so as to no longer abut container 12, the spring may push the distal surface of the flange of container 12, and thus the entire container 12, in a proximal direction. The inclusion of a spring may provide a lockout function to device 10 to prevent re-use of the device. One spring may surround container 12 in housing 14, or one or more springs may be positioned adjacent to container 12 and may abut a distal surface of a portion of container 12.

FIG. 2E depicts device 10 when thumb pad 20 initially abuts the proximal end of housing 14, prior to lockout of device 10, and FIG. 2F depicts device 10 once locked out, with plunger rod 16 and flange 26 seated in thumb pad 20, such that flange 26 abuts a distal interior wall 32 of thumb pad 20. Once plunger rod 16 and flange 26 are released from recess 23 of deflectable tab 22, and the distal end of container 12 is released from shoulder 28, plunger rod 16, flange 26, and container 12, may move in a proximal direction towards a distal interior wall 32 of thumb pad 20 (FIG. 2F).

In some embodiments, thumb pad 20 may include one or more arms, e.g., arms 36a and 36b that extend distally into housing 14 as thumb pad 20 is depressed. A distal end of arms 36a, 36b may protrude radially outwards away from container 12, and arms 36a, 36b may be biased radially outwards. Housing 14 may include a pair of cutouts 34a, 34b. When thumb pad 20 abuts the distal end of housing 14, releasing flange 26, plunger rod 16, and container 12, the protrusions on the distal ends of arms 36a, 36b may align with cutouts 34a, 34b and may deflect outwards to engage cutouts 34a, 34b (FIG. 2E). The engagement of the distal ends of arms 36a, 36b with cutouts 34a, 34b may maintain thumb pad 20 in place as flange 26 moves proximally to abut distal interior wall 32 of thumb pad 20 (FIG. 2F). It also serves to lock thumb pad 20 to housing 14, preventing re-use of device 10.

In some embodiments, multiple arms may extend distally from thumb pad 20. For example, thumb pad 20 may include two, three, four, or more arms 36a, 36b. In configurations with more than one arm, the arms may be equally spaced apart from one another. For example, if the thumb pad 20 includes four arms, each of the arms may be about 90 degrees from one another. If the thumb pad 20 includes two arms, the arms may be about 180 degrees from one another. Further, housing 14 may include a complimentary number of cutouts aligned with projections on the distal ends of the arms when thumb pad 20 is in a distal-most position. In some embodiments, a single arm may extend from thumb pad 20, and housing 14 may include a single cutout aligned with that arm. In some embodiments, thumb pad 20 may include two arms, and housing 14 may include two cutouts, one aligned with each arm, and so on. Although cutouts 34a, 34b are depicted as extending through the wall of housing 14, it is contemplated that cutouts 34a, 34b may be indentations or grooves that may not extend fully through housing 14. In embodiments in which the cutouts are grooves, one or more grooves may be positioned in line with the arms on thumb pad 20, or a single groove may extend around the inner wall of housing 14.

The embodiments of FIGS. 1A-2F depict a device with a passive safety mechanism that may activate when the user nears end-of-dose, i.e., when a substantial amount of the product is fully expelled from container 12. The passive safety mechanism of FIGS. 1A-2F may allow for container 12 and injection needle 2 (FIG. 1B) to move in a proximal direction into housing 14 at the end of the dose, so that injection needle 2 may be fully retracted in housing 14 to prevent accidental needle stick post-injection. Container 12 and injection needle 2 may not be moved proximally into housing 14 until injection is complete and thumb pad 20 is pressed against the proximal end of housing 14 to free flange 26 and container 12. As shown in FIGS. 1A-1B, only a portion of needle 2 may be seen by the user. This may prevent needle-phobia and needle-related anxiety as compared to conventional drug delivery devices with exposed needles. In addition, exposing only a portion of needle 2 may allow for usage of device 10 without any pre-injection steps, e.g., pinching of the skin to alter the injection depth to compensate for a needle length that is longer compared to the exposed needle 2 of device 10. Device 10 may be sized so that the starting position of container 12 within housing 14 is calibrated so that only a portion of the needle is exposed, and the length of the exposed portion of needle 2 represents the desired insertion depth of needle 2.

In some embodiments, device 10 may include a feedback mechanism. Device 10 may include a visual feedback mechanism, e.g., thumb pad 20 and housing 14 being locked together and needle 2 being retracted into housing 14, so needle 2 cannot be seen by user once device 10 is pulled away from the injection site. In some examples, device 10 may include an optional opening 40 (FIG. 1B) for viewing container 12 and movement of plunger rod 16 and stopper 18 in container 12. In some aspects, device 10 may include an audible feedback mechanism, e.g., an audible “clicking” sound once thumb pad 20 and housing 14 connect and/or once flange 26 abuts distal interior wall 32 of thumb pad 20. In some aspects, device 10 may include a tactile feedback, e.g., a snap or vibration as thumb pad 20 and housing 14 connect and/or once flange 26 abuts distal interior wall 32 of thumb pad 20.

Device 10 may be of any suitable size and shape to hold or partially hold a product container 12, and/or to support and hold plunger rod 16 and stopper 18 in product container 12. Thumb pad 20 may be of any suitable size and shape to abut and lock into device 10. Components of thumb pad 20, including deflectable tab 22 and recess 23, may be of any suitable size and shape to receive flange 26. Cutouts 34a, 34b, may be of any suitable size and shape to receive arms 36a, 36b.

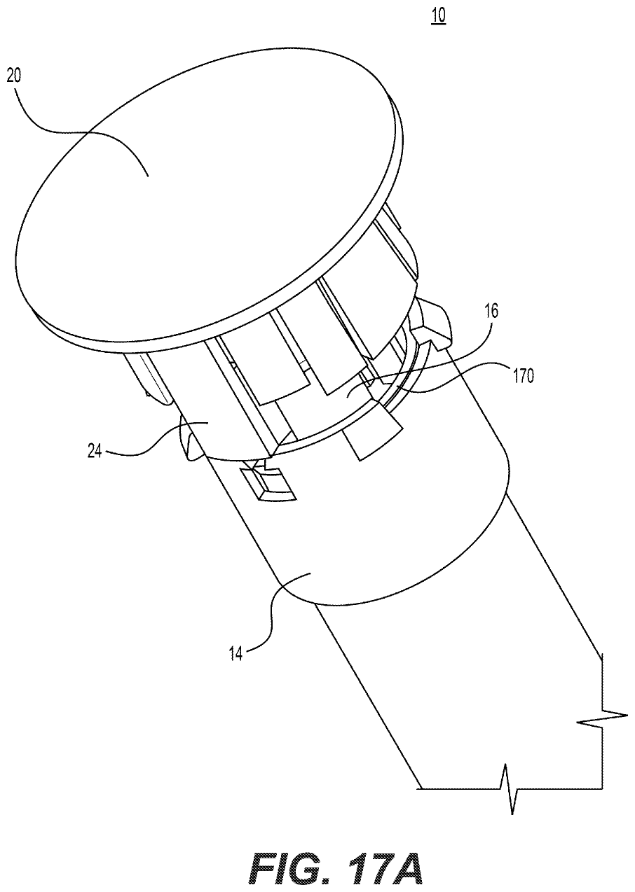

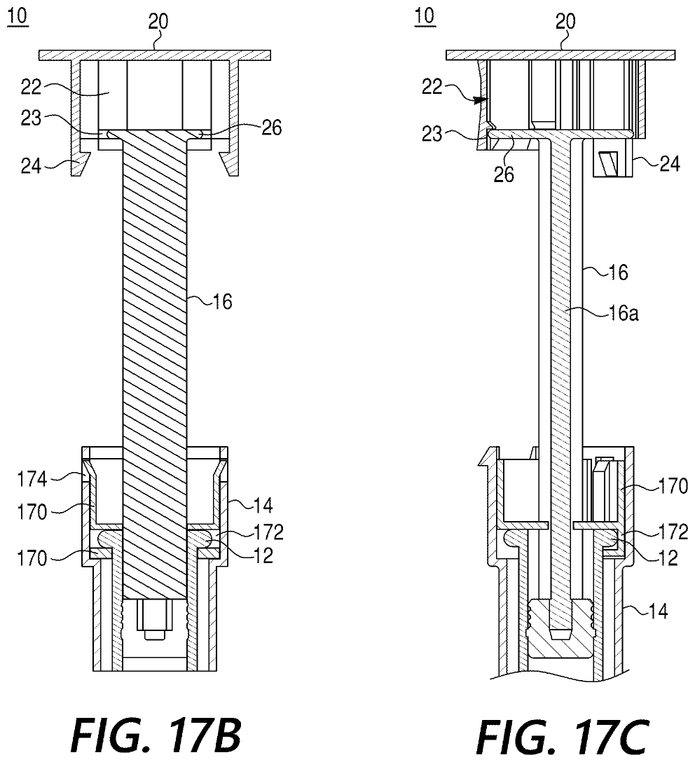

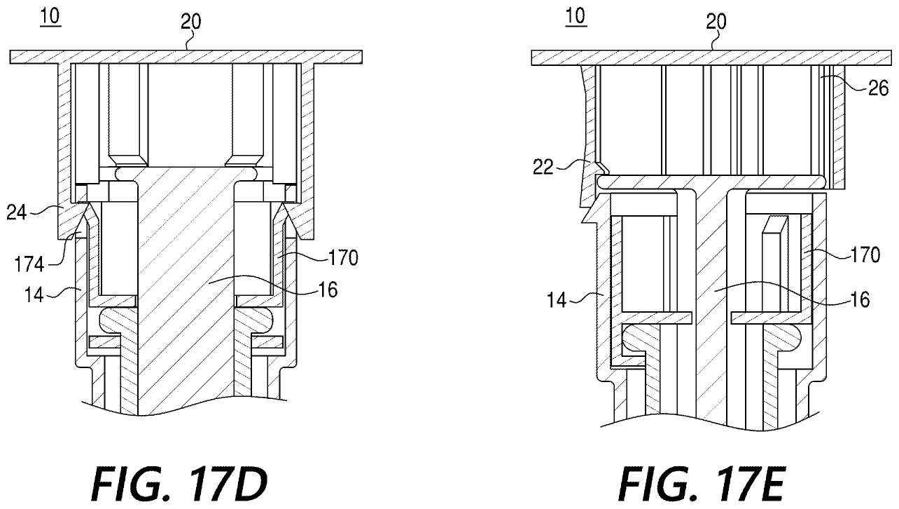

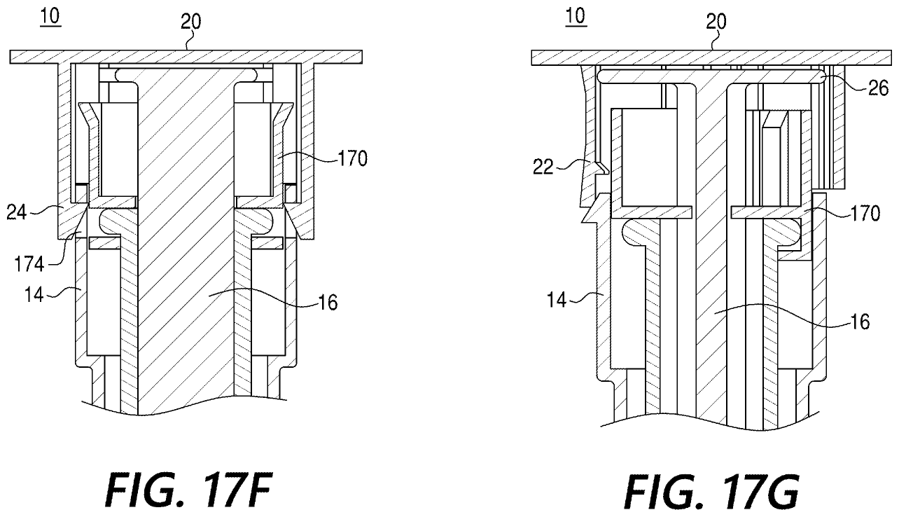

In other embodiments, device 10 may include a rear cap 170 (FIGS. 17A and 17B). As shown in FIG. 17B, rear cap 170 may be shaped and configured to fit into a proximal end of housing 14. Rear cap 170 may have a substantially circular shape. A proximal end of rear cap 170 may abut a proximal end of housing 14 such that rear cap 170 may be secured in housing 14. Referring to FIG. 17B, rear cap 170 may include a rear cap recess 172 for receiving a portion of syringe 12. For example, a proximal portion of syringe 12 may fit into rear cap recess 172 of rear cap 170. Thumb pad 20 of device 10 may include deflectable tab 22. In some examples, thumb pad 20 may include a plurality of deflectable tabs 22. Deflectable tab 22 may include a recess 23 configured to receive a flange 26 of plunger rod 16. Thumb pad 20 may also include a proximal extension 24. In some examples, thumb pad 20 may include a plurality of proximal extensions 24. FIG. 17C depicts a rotated view of device 10 in FIG. 17B. Referring to FIG. 17C, plunger rod 16 may include a plurality of ridges 16a, wherein portions of rear cap 170 may abut against portions of ridges 16.

During use, the user may press thumb pad 20 in a distal direction towards housing 14. Similar to the mechanism described in reference to FIGS. 2A and 2B, and referring to FIGS. 17D and 17E, as thumb pad 20 is pushed towards housing 14, a portion at the proximal end of housing 14 may abut deflectable tabs 22. Deflectable tabs 22 may deflect in a direction away from flange 26, allowing plunger rod 16 and thumb pad 20 to decouple. FIG. 17E depicts a rotated view of device 10 in FIG. 17D. Referring to FIG. 17F, at end-of-dose, a distal end of extensions 24 may click into housing 14. For example, housing 14 may include cutouts 174 for receiving the distal end of extensions 24. Once the distal end of extensions 24 click into cutouts 174, the distal end of extensions 24 may press a portion of rear cap 170 inwards towards plunger rod 16, allowing rear cap 170 to move into a proximal direction into thumb pad 20. Device 10 may include an actuator, e.g., a spring, (not shown) underneath rear cap 170. The actuator may be configured to push rear cap 170 into thumb pad 20 once a portion of rear cap 170 is depressed inwards towards plunger rod 16. FIG. 17G depicts a rotated view of device 10 in FIG. 17F.

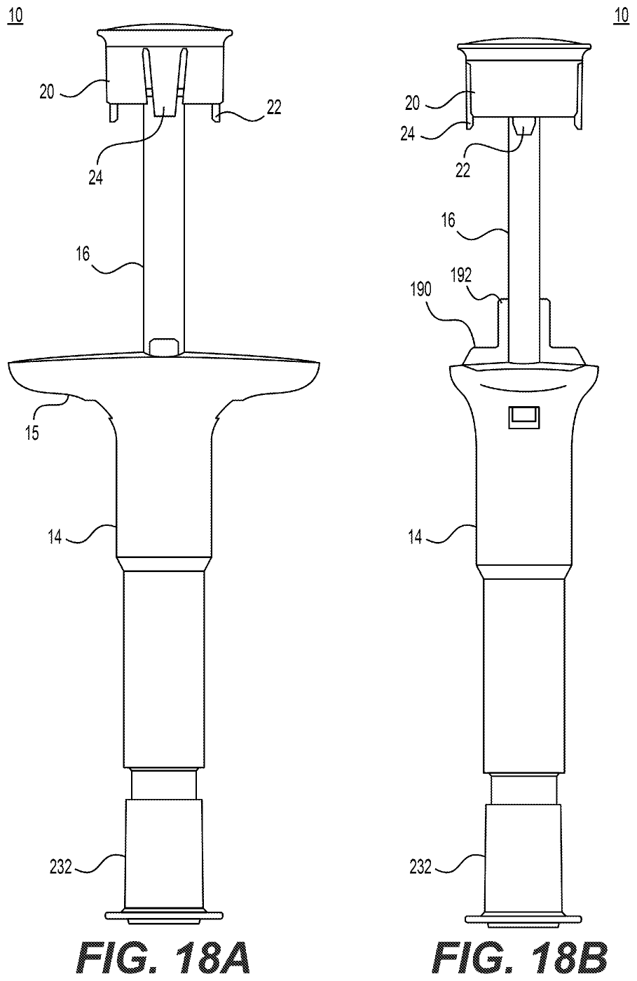

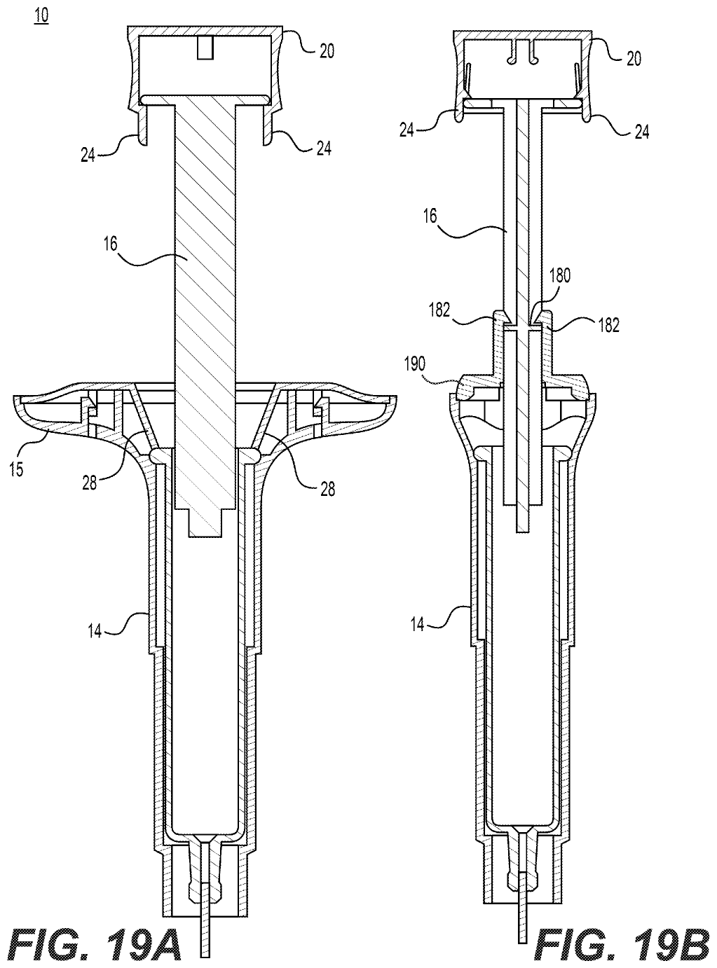

FIGS. 18A and 18B depict alternative embodiments of device 10. FIG. 18A depicts a frontal exterior view of device 10 and FIG. 18B depicts a side exterior view of device 10. Housing 14 of device 10 may include a finger flange 15. Finger flange 15 may be located at a proximal end of housing 14 and extend in a direction away from plunger rod 16. Finger flange 15 may be shaped or configured to be properly held by a user and properly operable with a syringe. For example, finger flange 15 may be designed to meet safety requirements and requisite needle distance requirements. Referring to FIG. 18B, housing 14 may include a rear cap 190. In this embodiment, rear cap 190 may be located at a proximal end of housing 14. Rear cap 190 may include a hook portion 192 extending in a proximal direction. As shown in FIG. 18B, rear cap 190 may include at least two hook portions 192. Hook portions 192 may sit against a recessed portion 180 of plunger rod 16 (shown in detail in FIGS. 19A and 19B). Hook portions 192 may hook onto, i.e., sit atop, a proximal surface of recessed portion 180.

FIGS. 19A and 19B depict cross-sectional views of device 10 in FIGS. 18A and 18B. FIG. 19A is a front cross-sectional view of device 10 and FIG. 19B is a side cross-sectional view of device 10. Cap 20 may include proximal extensions 24 and housing 14 may include finger flange 15. As discussed above, finger flange 15 may include rear cap 190, where rear cap 190 may include hook portions 182 that may hook onto recessed portions 180 of plunger rod 26. Referring to FIG. 19A, housing 14 may also include shoulder 28. Further details of housing 14 and rear cap 190 will be discussed below.

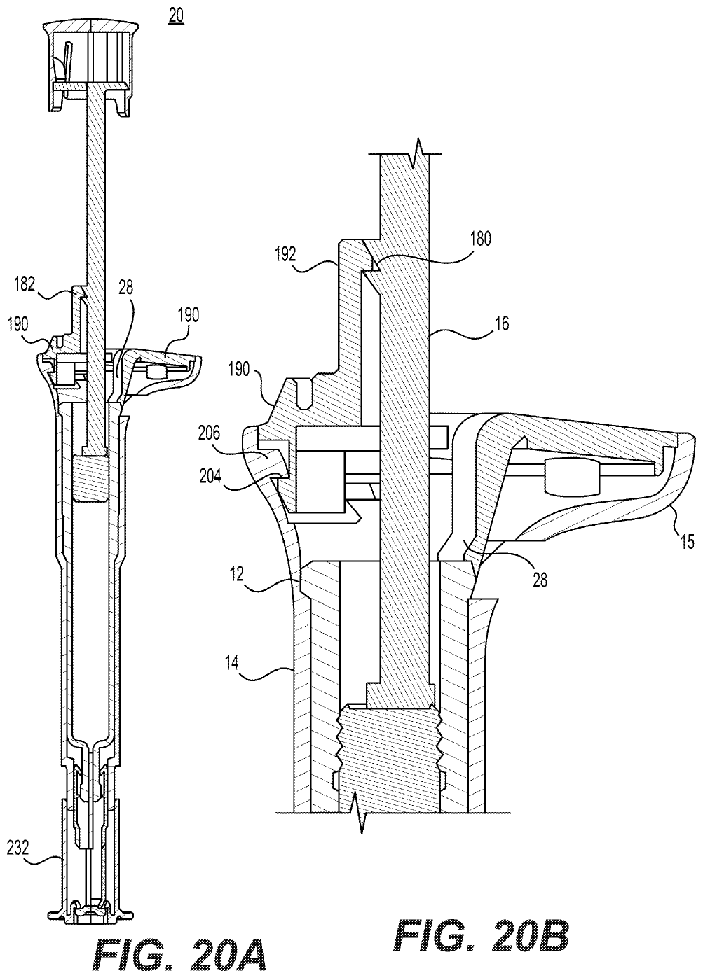

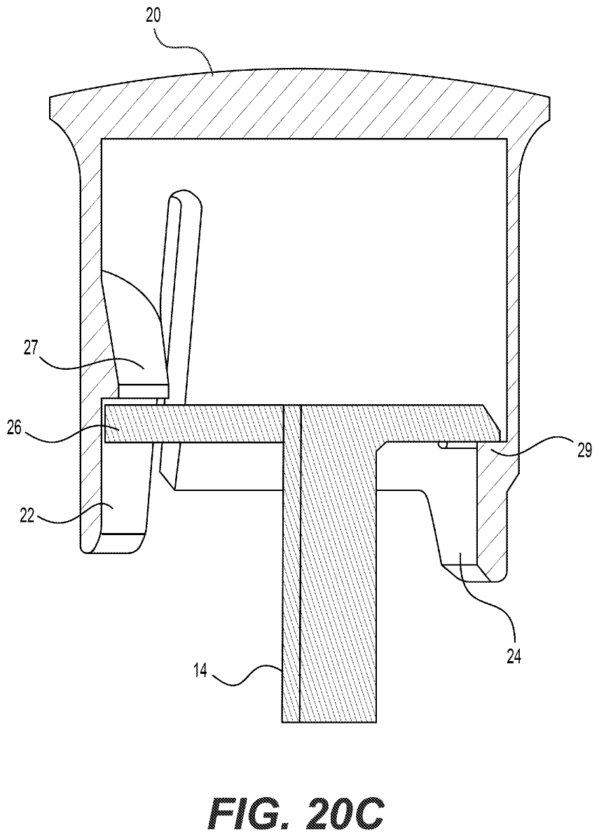

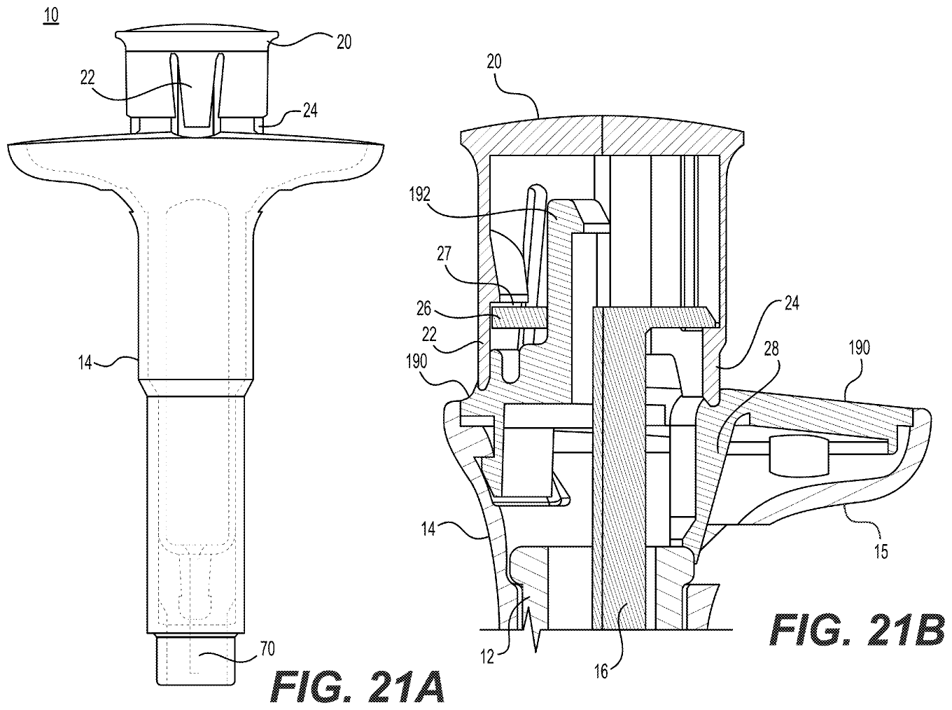

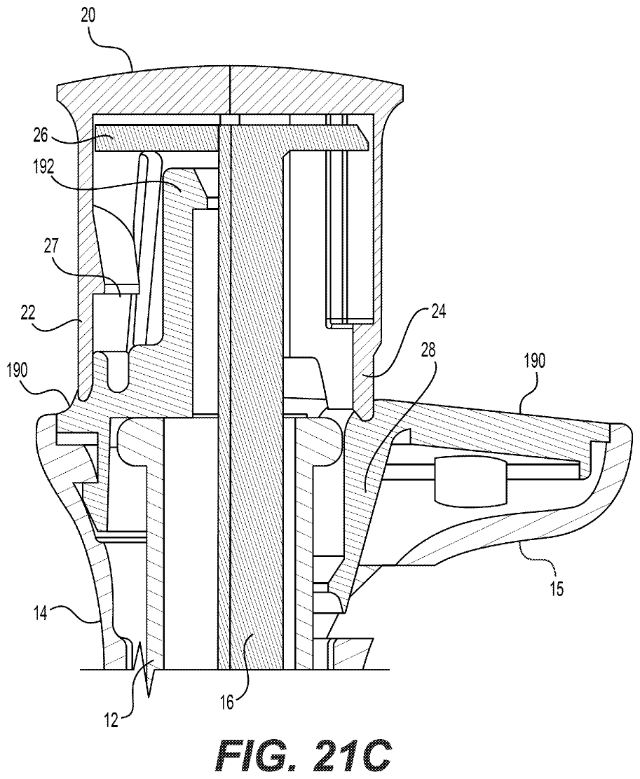

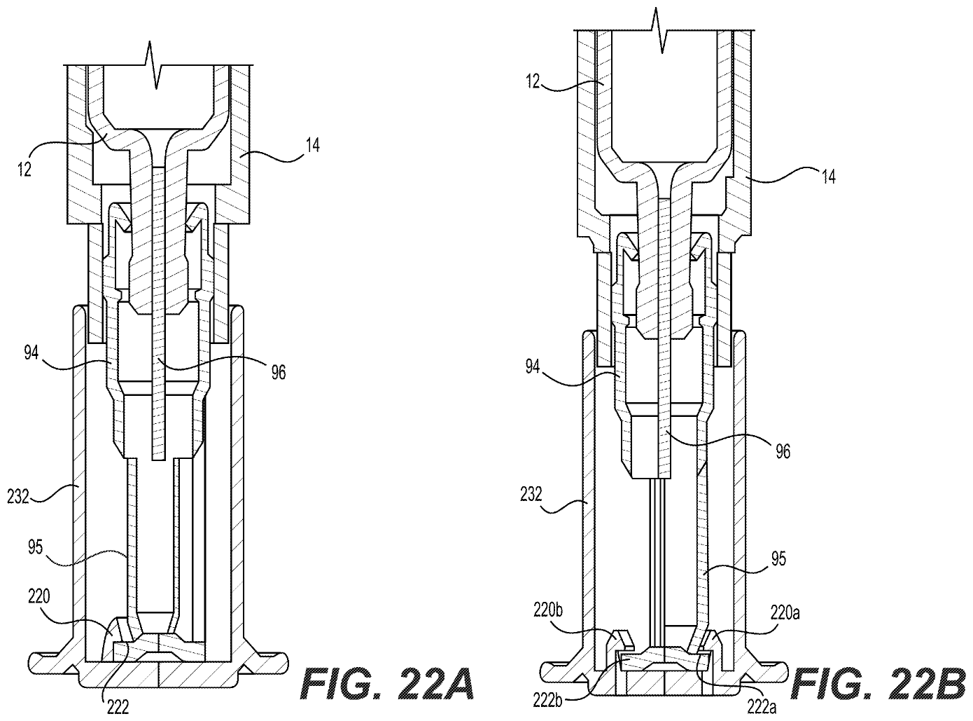

FIGS. 20A-20C depict views of device 10 in a pre-injection state. FIG. 20A depicts a quarter cross-sectional view of device 10, FIG. 20B depicts a detailed view of the proximal end of housing 14 and rear cap 190 from FIG. 20A, and FIG. 20C depicts a detailed view of thumb pad 20 from FIG. 20A. Referring to FIG. 20B, rear cap 190 and housing 14 may include attachment means for attaching rear cap 190 to housing 14. For example, rear cap 190 may include a snap 201 and housing 14 may include an insert (not shown), allowing rear cap 190 and housing 14 to snap together. Rear cap 190 may include tabs 203 and housing 14 may include housing hooks 205. When rear cap 190 and housing 14 are attached (FIG. 20B), tabs 203 may sit into a portion of housing 14 and housing hooks 205 may hook onto, i.e., sit atop, a portion of tabs 203. This configuration may serve as a pull-back prevention feature preventing the user from inadvertently pulling plunger rod 14 out of the back of device 10. This configuration may also serve as a pre-expulsion prevention to prevent the loss of drug product before the needle is inserted into the skin. As shown in FIG. 20C, thumb pad 20 may include deflectable tab 22 and proximal extension 24. FIG. 20C depicts a quarter cross-sectional view of thumb pad 20 from FIG. 20A. Thumb pad 20 may include a plurality of deflectable tabs 22 and proximal extensions 24. Referring to FIG. 20C, a portion of flange 26 of plunger rod 14 abuts a distal surface of indent 27 of deflectable tab 22. Further, a portion of flange 26 may also sit atop a proximal surface of ledge 29 of distal extension 24. FIGS. 21A-21C depict device 10 in FIGS. 18A-20C as it transitions to a post-injection, i.e., end-of-dose state. At the start of injection, the needle of syringe 12 (not shown) may extend distally from housing 14. For example, about 6 mm of the needle may be exposed at the start of injection. While the user presses thumb pad 20 in a distal direction towards housing 14, plunger rod 16 may also extend distally into syringe 12, allowing for recessed portion 180 to detach from hook portion 192. Referring to FIG. 21B, once thumb pad 20 abuts rear cap 190, deflectable tab 22 may deflect outwards in a direction away from flange 26, allowing deflectable tab 22 to detach from flange 26. Once thumb pad 20 abuts rear cap 190, distal extension 24 may abut against shoulder 28, pushing shoulder 28 outwards in a direction away from syringe 12. Syringe 12 and plunger rod 16 may then move into a proximal direction, such that flange 26 of plunger rod 16 may abut a distal surface of thumb pad 20 (FIG. 21C). The needle 70 of syringe 12 may then be shrouded within housing 14 (FIG. 21A). This passive end-of-dose lockout mechanism may be activated by an actuator, e.g., spring 13, that may be located around a proximal end of syringe 12 (FIG. 1B).

Embodiments of device 10 depicted in FIGS. 18A-21C may include one or more additional features, e.g., features to lock thumb pad 20 in place with plunger rod 16 at a post-injection state; alignment features between thumb pad 20 and plunger rod 16 to reduce misalignment at the end of injection; ergonomic design to enable two hand operation, i.e., one hand may hold the syringe for needle insertion at 90 degrees, while the second hand pushes plunger rod 16; optimal configuration and sizing of deflectable tab 22, distal extension 24, plunger rod 16, and/or rear cap 190; indicators of injection completion, e.g., a visual color change or audible indicator; activation points to mitigate potential failure modes, e.g., a pre-activation point or state; optimal configuration and sizing of thumb pad 20 to fit a user's hand; extension of plunger rod 16 to sit within a piston of the device to improve alignment; and optimization of the design to reduce the required spring force to minimize plastic creep.

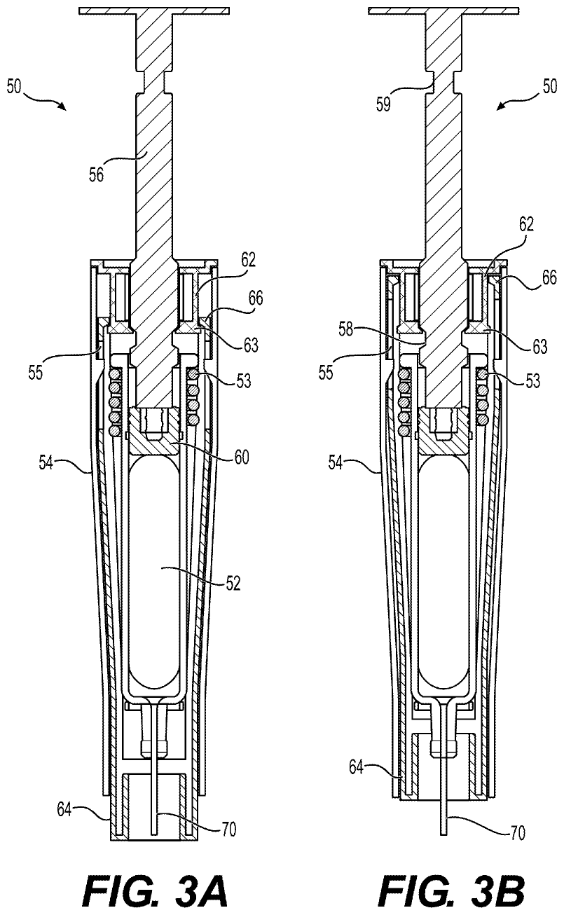

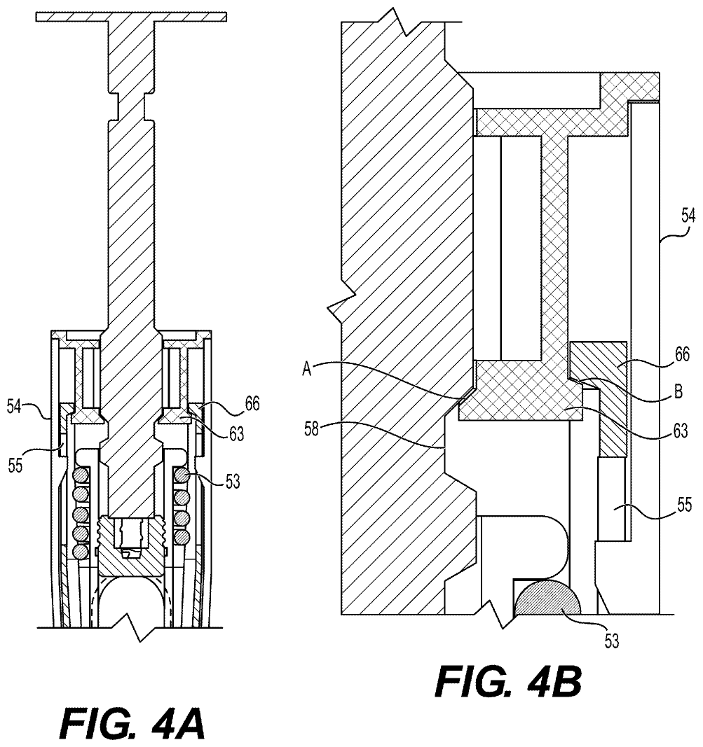

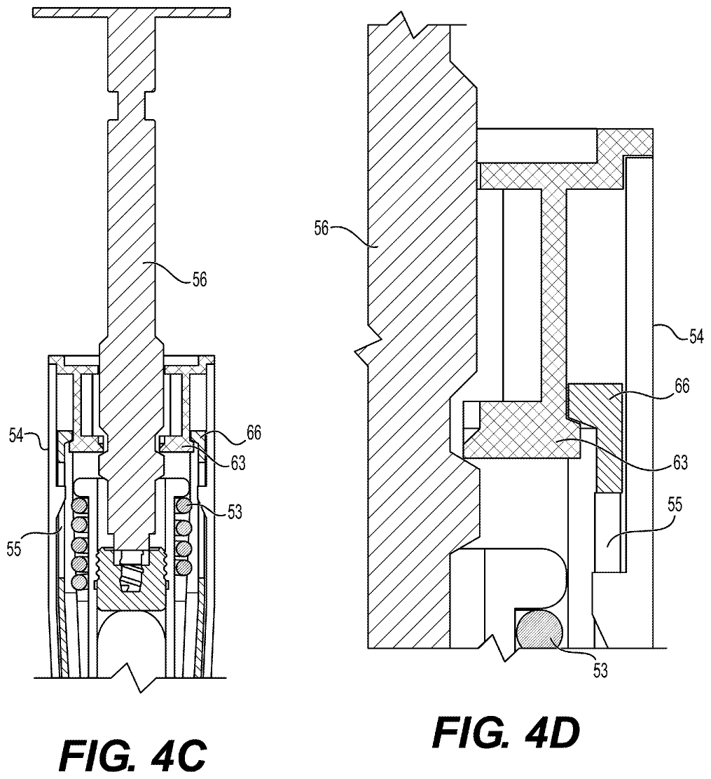

FIGS. 3A-5D depict another embodiment of the present disclosure in which the needle is covered in a pre-injection state. Referring to FIGS. 3A-3B, device 50 may be designed to hold any conventional product container 52, e.g., a syringe. Device 50 may include a housing 54, plunger rod 56, stopper 60, blocking component 62, and needle cover 64. As shown, for example, in FIG. 3B, plunger rod 56 may include a first indent 58 at a distal region and a second indent 59 at a proximal region. Blocking component 62 may be located at a proximal end of housing 54. Plunger rod 56 may be slidably coupled to blocking component 62, such that plunger rod 56 may slide in a distal direction through blocking component 62. Needle cover 64 may include a proximal end 66 abutting a deflectable arm 63 of blocking component 62. As shown in FIGS. 3A-3B, blocking component 62 may include more than one deflectable arm 63. For example, blocking component 62 may include two, three, four, or more deflectable arms 63. In configurations with more than one deflectable arm, the deflectable arms may be equally spaced apart from one another. For example, if the blocking component 62 includes four deflectable arms 63, each of the deflectable arms may be about 90 degrees from one another. For example, blocking component 62 may have a pair of deflectable arms 63 spaced, e.g., about 180 degrees from one another. Accordingly, the discussion herein may be applicable to a device with a varying number of components. In some examples, needle cover 64 may include a slot 55 (FIGS. 3A-4D) configured for receiving a portion of housing 54. Slot 55 will be further described below.

FIG. 3A depicts device 50 prior to use, where needle cover 64 may be fully extended distally of housing 54 and covers needle 70 in an initial, starting position. FIG. 3B depicts device 50 during use, once needle cover 64 may be depressed and moved proximally into housing 54, e.g., by pressing device 50 against the injection site. Detailed configurations and interactions of needle cover 64 and blocking component 62 will be further described as the use of device 50 is discussed.

At an initial position, prior to use, as shown in FIGS. 4A-4B, a distal end of deflectable arm 63 may fit into a portion of first indent 58 of plunger rod 56. For example, a distal portion of deflectable arm 63 may abut a portion of first indent 58 at contact point A. Distal end 66 of needle cover 64 may also abut a portion of deflectable arm 63 at a contact point B. At this initial position, prior to use, significant motion of plunger rod 56 in either a proximal or a distal direction may be prevented. For example, as shown in FIGS. 4A-4B, if plunger rod 56 is pushed in a distal direction into housing 54, deflectable arm 63 is prevented from deflecting outward by proximal end 66 of needle cover 64, locking deflectable arm 63 within first indent 58 and preventing further movement of plunger rod 56. As shown in FIGS. 4C-4D, if plunger rod 56 is pulled in a proximal direction away from housing 54, deflectable arm 63 is again prevented from deflecting outward by proximal end 66 of needle cover 64, locking deflectable arm 63 within first indent 58 and preventing further movement of plunger rod 56.

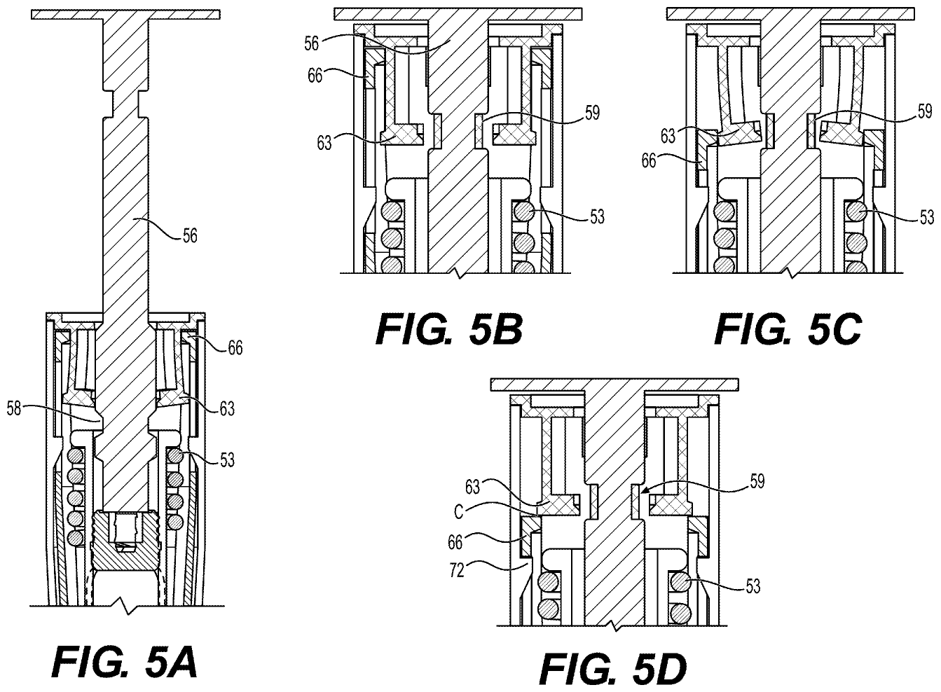

To initiate injection, device 50 may be pressed against an injection site to depress needle cover 64. Once needle cover 64 is moved distally, proximal end 66 of needle cover 64 moves proximally away from engagement with deflectable arm 63. This may allow plunger rod 56 to be pressed in a distal direction towards housing 54 for injection. In other words, as needle cover 64 moves proximally into housing 54 and proximal end 66 moves away from contact point B, plunger rod 56 may deflect arm 63 of blocking component 62, allowing first indent 58 to move past deflectable arm 63 (FIG. 5A) so that injection can proceed. As shown in FIG. 5A, proximal end 66 of needle cover 64 moves proximally in the housing and may abut a portion of blocking component 62.

Once injection is complete, device 50 may be removed from the injection site and needle cover 64 may be pulled downwards in a distal direction (FIGS. 5B-5C). In some examples, device 50 may include a spring shroud 53 (FIGS. 3A and 5A-5D) to pull needle cover 64 and bias needle cover 64 in a proximal direction. As show in FIGS. 5B-5C, plunger rod 56 is fully depressed into housing 54. As shown in FIGS. 4A and 4C, the portion of plunger rod 56 at second indent 59 may be smaller in diameter compared to the portion of plunger rod 56 at first indent 58, creating a larger second indent 59 compared to first indent 58. As such, once plunger rod 56 is fully depressed into housing 54 at the end of injection, there is an increased area between plunger rod 56 and deflectable arm 63 at second indent 59 (FIG. 5B). This space may allow for distal end 66 of needle cover 64 to flex deflectable arm 63 inwards towards plunger rod 56, so that proximal end 66 may move past deflectable arm 63 in a distal direction (FIG. 5C). In some examples, there may be a spring shroud 53 around needle cover 64, wherein needle cover 64 may move past deflectable arm 63 using spring force. Referring to FIG. 5D, once proximal end 66 has passed deflectable arm 63, deflectable arm 63 may return to an un-deflected position, and proximal end 66 may be stopped from moving in a proximal direction again by deflectable arm 63. Deflectable arm 63 may now act as a lockout mechanism to prevent re-use of device 50 or accidental needle stick, since needle cover 64 can no longer be moved proximally to expose the needle 70. At this final, post-injection position of device 50, needle cover 64 may be in a lockout position, and proximal end 66 and deflectable arm 63 may abut at contact point C (FIG. 5D). Needle cover 64 and proximal end 66 may be prevented from further movement, with proximal end 66 being held between ledge 72 of housing 54 and deflectable arm 63. Additionally, device 50 may not be re-used.

In some embodiments, slot 55 of needle cover 64 may be configured to slidably receive ledge 72. For example, as needle cover 64 moves, ledge 72 may move within slot 55. Ledge 72 may move in a distal and/or proximal direction within slot 55. As described above, at the post-injection position of device 50, proximal end 66 may be held between ledge 72 of housing 54 and deflectable arm 63, wherein ledge 72 may be within slot 55. As shown in FIG. 3A, needle cover 64 may include a complimentary number of slots aligned with ledges 72 of housing 54. In some embodiments, housing 54 may include a single ledge and needle cover 64 may include a single slot aligned with that ledge. In some embodiments, housing 54 may include two ledges and needle cover 64 may include two slots, one aligned with each ledge, and so on. Slot 55 may extend through needle cover 64. In other embodiments, slot 55 may be an indentation or groove that may not extend fully through needle cover 64.

The embodiments of FIGS. 3A-5D depict a device with a passive safety mechanism that may activate when the user nears end-of-dose, i.e., when a substantial amount of the product is fully expelled from container 52. The passive safety mechanism of FIGS. 3A-5D may prevent accidental post-injection needle stick, as needle cover 64 may not be depressed until device 50 is pressed against the injection site, and once device 50 is removed from the injection site, needle cover 64 springs back to extend from the distal end of housing 54 to cover needle 70.

Additionally, the passive safety mechanism of FIGS. 3A-5D may prevent premature lockout. For example, some conventional drug delivery devices may automatically lockout once the device is removed from the injection site, even if the product has not been fully expelled. Referring to FIGS. 5C-5D, only when plunger rod 56 is fully depressed into housing 54, can proximal end 66 of needle cover 64 flex deflectable arm 63 into second indent 59, so that proximal end 66 may move in a distal direction past deflectable arm 63 to the position adjacent ledge 72 to abut deflectable arm 63 at contact point C. Plunger rod 56 is only fully depressed at the end of the dose, and thus lockout may not occur prior to the entire dose of the product in container 52 having been expelled. If device 50 is removed from the injection during the injection, i.e., prior to plunger rod 56 being fully depressed into housing 54, needle cover 64 may not be locked out, and device 50 may be placed back onto the injection site to continue the injection.

Additionally, as needle 70 is within needle cover 64 before and after injection, the user may not see needle 70 at any point prior, during, and/or post injection. Needle cover 64 may thus reduce needle-phobia and needle-related anxiety as compared to conventional drug delivery devices with exposed needles. In addition, exposing only a portion of needle 70 may allow for usage of device 50 without any pre-injection steps, e.g., pinching the skin to alter the injection depth to compensation for a needle length that is longer compared to the exposed needle 70 of device 50 (FIG. 3B). Device 50 may be configured, e.g., container 52 may be positioned within housing 54 and/or needle cover 64 may be sized, so that the retracted position of needle cover 64 exposes only a portion of needle 70, and the length of the exposed portion of needle 70 represents the desired insertion depth of needle 70.

In some embodiments, device 50 may include a feedback mechanism. Device 50 may include a visual feedback mechanism, e.g., plunger rod 56 fully depressed into housing 54 (FIG. 5B) and needle cover 64 fully extended proximal to housing 54 with needle 70 covered. In some examples, device 50 may include an opening for viewing container 52 and movement of plunger rod 56 and stopper 60 in container 52. Device 50 may also include an audible feedback mechanism, e.g., an audible “clicking” sound once a proximal end of plunger rod 56 and housing 54 connect. In some aspects, device 50 may include a tactile feedback, e.g., a snap or vibration as needle cover 64 moves proximally and abuts ledge 72 at the end of the injection.

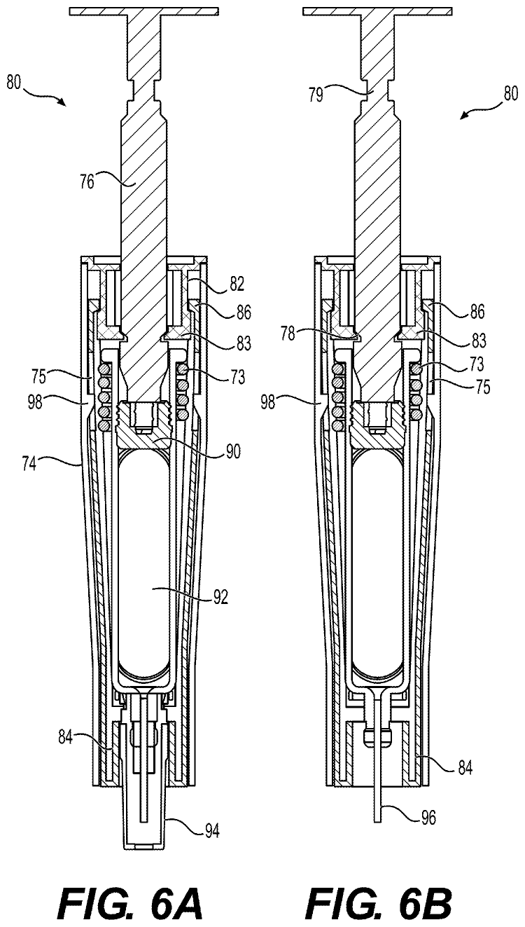

FIGS. 6A-6B depict another embodiment of the present disclosure, similar to those described above in reference to FIGS. 3A-5D, but in which the needle extends beyond the needle cover in a pre-injection state. Where appropriate, features will be designated with references similar to those discussed above. Any features and components that correspond to those in FIGS. 3A-5D may be understood to be configured similarly. Referring to FIGS. 6A-6B, device 80 may be designed to hold any conventional product container 92, e.g., a syringe. Device 80 may include a housing 74, plunger rod 76, stopper 90, blocking component 82, and needle cover 84. As shown, for example, in FIG. 6B, plunger rod 76 may include a first indent 78 at a distal region and a second indent 79 at a proximal region. Blocking component 82 may be located at a proximal end of housing 74. Plunger rod 76 may be slidably coupled to blocking component 82, such that plunger rod 76 may slide through blocking component 82. As shown in FIGS. 6A-6B, blocking component 82 may include more than one deflectable arm 83. For example, blocking component 82 may include two, three, four, or more deflectable arms 83. In configurations with more than one deflectable arm, the deflectable arms may be equally spaced apart from one another. For example, if the blocking component 82 includes four deflectable arms 63, each of the deflectable arms may be about 90 degrees from one another. For example, blocking component 82 may have a pair of deflectable arms 83 spaced, e.g., about 180 degrees from one another. Accordingly, the discussion herein may be applicable to a device with a varying number of components.

FIG. 6A depicts device 80 prior to use, where needle 96 extends distally from housing 74 and needle cover 84. An optional removable needle shield 94 may be inserted into or onto needle cover 84 and/or housing 74, to cover needle 96. FIG. 6B depicts device 80 prior to use, once needle shield 94 is removed. In contrast to the embodiments of FIGS. 3A-3B, since needle 96 extends beyond needle cover 84 in the device's initial state, the needle cover 84 may not need to be depressed prior to use. Once needle shield 94 is removed, needle 96 may be visible to a user prior to injection.

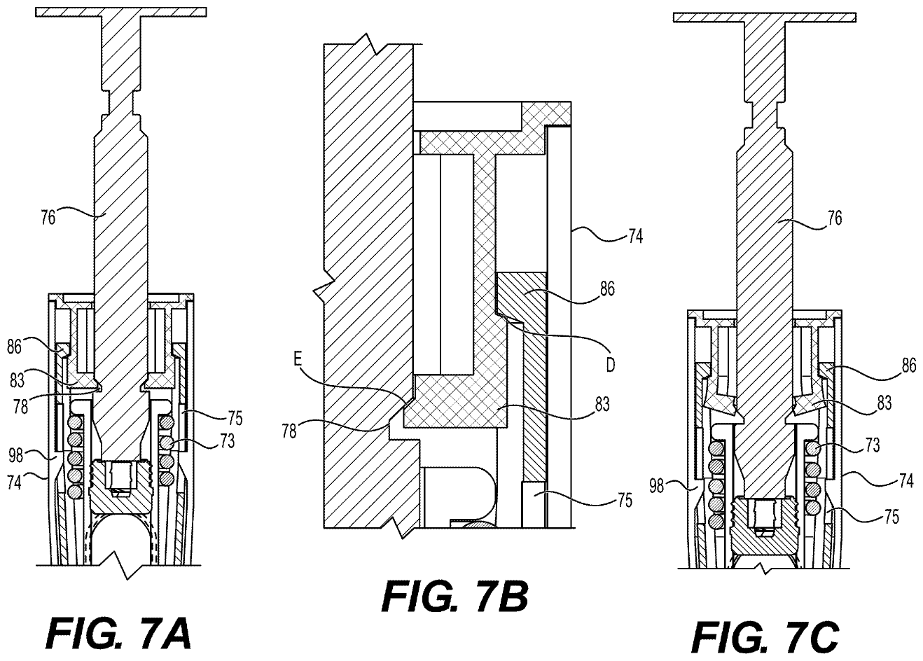

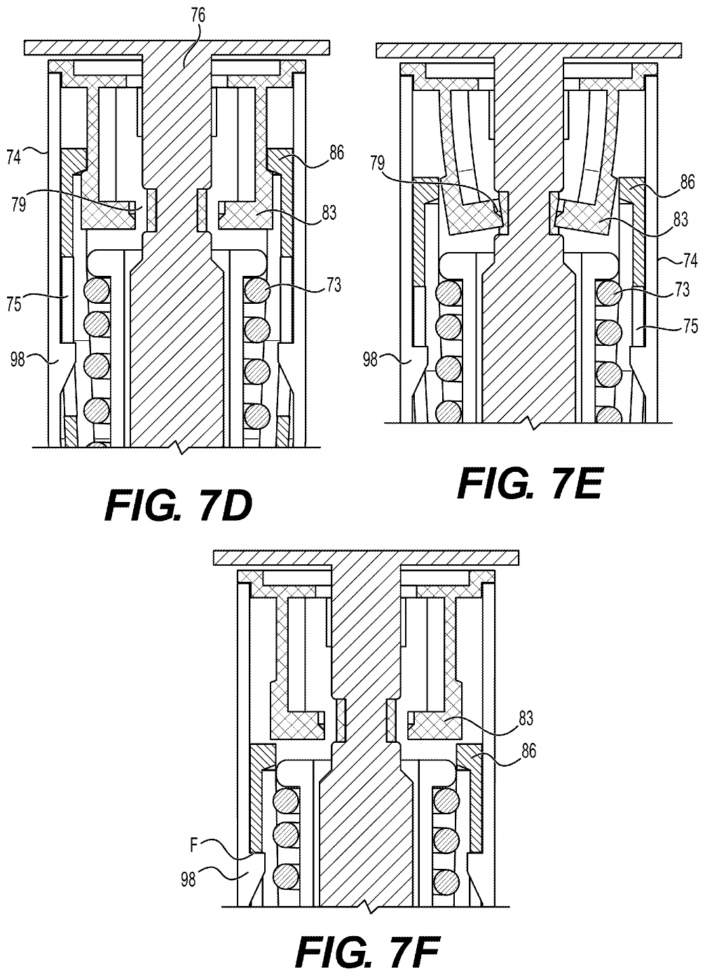

At an initial position, prior to use, as shown in FIGS. 7A-7B, a distal end of deflectable arm 83 may fit into at least a portion of first indent 78 of plunger rod 76. For example, a distal portion of deflectable arm 83 may abut a portion of first indent 78 at contact point E. Proximal end 86 of needle cover 84 may also abut a portion of deflectable arm 83 at a contact point D. At this initial position, prior to use, significant motion of plunger rod 76 in either a proximal or a distal direction may be prevented. For example, if plunger rod 76 is pushed in a distal direction into housing 74 using a normal injection force, contact between deflectable arm 83 and plunger rod 76 at contact point E, as well as the contact between proximal end 86 of needle cover 84, may prevent proximal movement of plunger rod 76. The arrangement of deflectable arm 83, plunger rod 76 with first indent 78, and proximal end 86 of needle cover 84 may prevent movement of plunger rod 76 in a distal direction unless an increased amount of force is applied, i.e., a force higher than a typical injection force. If plunger rod 76 is pulled in a proximal direction away from housing 74, deflectable arm 83 may contact an edge of first indent 78, preventing movement of plunger rod 76 in a distal direction.

To initiate injection, device 80 may be pressed against an injection site, allowing needle 96 to pierce injection site. Plunger rod 76 may then be pushed in a distal direction towards housing 74 using a force higher than normal injection force. As plunger rod 76 is depressed into housing 74, deflectable arm 83 may be deflected radially outwards in a direction away from plunger rod 76 and contact point E when subjected to the higher than normal break loose force, allowing first indent 78 to move past deflectable arm 83 (FIG. 7C). A higher than normal break loose force may be, for example, a force above about 2 Newtons (N), e.g., about 3 N to about 20 N.

Once injection is complete, device 80 may be removed from the injection site, and as shown in FIG. 7D, plunger rod 76 may be fully depressed into housing 74, such that plunger rod 76 may abut housing 74. Once device 80 is removed from the injection site, needle cover 84 may include a spring shroud 73 (FIG. 6A) to move needle cover 84 downwards in a distal direction. As shown in FIGS. 6A-6B, the portion of plunger rod 76 at second indent 79 may be smaller in diameter compared to the portion of plunger rod 76 at first indent 78, creating a larger second indent 79 compared to first indent 78. As such, once plunger rod 76 is fully depressed into housing 74 at the end of injection, there is an increased area between plunger rod 76 and deflectable arm 83 at second indent 79 (FIG. 7D). This space may allow for proximal end 86 of needle cover 84 to flex deflectable arm 83 inwards towards plunger rod 76, so that proximal end 86 may move past deflectable arm 83 in a distal direction (FIG. 7E). In some examples, there may be a spring shroud (not shown) around needle cover 84 or at an end of needle cover 84, wherein needle cover 84 may move past deflectable arm 83 using spring force. Referring to FIG. 7E, once distal end 86 has passed deflectable arm 83, deflectable arm 83 may flex back to its initial position, and needle cover 84 may be stopped from moving in a proximal direction again by deflectable arm 83. Deflectable arm 83 may now act as a lockout mechanism to prevent re-use of device 80 or accidental needle stick, since needle cover 84 may extend further distally compared to the starting position to that needle cover 84 covers needle 96. At this final, post-injection position of device 80, needle cover 84 may be extended distally in a lockout position, and needle cover 84 may abut ledge 98 at a contact point F, preventing further movement of needle cover 84 in a proximal direction. Proximal end 86 of needle cover 84 may also abut a distal end of deflectable arm 83 such that further proximal movement of needle cover 84 and proximal end 86 may be prevented.

In some examples, needle cover 84 may include a slot 75 (FIG. 6A) configured for receiving a portion of housing 74. In some embodiments, slot 75 of needle cover 84 may be configured to slidably receive ledge 98. For example, as needle cover 84 moves, ledge 98 may move within slot 75. Ledge 98 may move in a distal and/or proximal direction within slot 75. As described above, at the post-injection position of device 80, proximal end 86 may be held between ledge 98 of housing 74 and deflectable arm 83, wherein ledge 98 may be within slot 75. As shown in FIG. 6A, needle cover 84 may include a complimentary number of slots aligned with ledges 98 of housing 74. In some embodiments, housing 74 may include a single ledge and needle cover 84 may include a single slot aligned with that ledge. In some embodiments, housing 74 may include two ledges and needle cover 84 may include two slots, one aligned with each ledge, and so on. Slot 75 may extend through needle cover 84. In other embodiments, slot 75 may be an indentation or groove that may not extend fully through needle cover 84.

The embodiments of FIGS. 6A-7F depict a device with a passive safety mechanism that may activate when the user nears end-of-dose, i.e., when a substantial amount of the product is fully expelled from container 92. The passive safety mechanism of FIGS. 6A-7F may also prevent premature lockout. For example, some conventional drug delivery devices may automatically lock out once the device is removed from the injection site, even if the product has not been fully expelled. Referring to FIGS. 7D-7F, only when plunger rod 76 is fully depressed into housing 74, can proximal end 86 of needle cover 84 flex deflectable arm 83 into second indent 79, so that needle cover 84 may move in a distal direction past deflectable arm 83 and then abut ledge 98 at contact point F. Once plunger rod 76 is fully depressed, the entire dose of the product in container 92 may be expelled. If device 80 may be removed from the injection site during the injection, i.e., prior to plunger rod 76 being fully depressed into housing 74, needle cover 84 may not be locked out, and device 80 may be placed back onto the injection site to continue the injection.

Additionally, as a portion of needle 96 is within needle cover 84, the user may only see a portion of needle 96 at any point prior to and/or during injection. As needle cover 84 may extend from a proximal end of housing 74 to cover needle 96 post-injection, the user may not see any portion of needle 96 at end-of-dose. Needle cover 84 may thus reduce needle-phobia and needle-related anxiety as compared to conventional drug delivery devices with exposed needles. In addition, exposing only a portion of needle 96 may allow for usage of device 80 without any pre-injection steps, e.g., pinching the skin to alter the injection depth to compensation for a needle length that is longer compared to the exposed needle 96 of device 80 (FIG. 6B). Device 80 may be configured, e.g., container 92 may be positioned within housing 74 and/or needle cover 84 may be sized, so that needle cover 84 exposes only a portion of needle 96, and the length of the exposed portion of needle 96 represents the desired insertion depth of needle 96.

Referring to FIG. 7C, the safety mechanism may also prevent pre-expulsion of the product. As described above, the force exerted by deflectable arm 83 and proximal end 86 of needle cover 84 on plunger rod 76 prevents movement of plunger rod 76 until an initial “break” force, i.e., a greater than normal injection force, is produced to overcome the force between deflectable arm 83, proximal end 86, and plunger rod 76. The “break” force can be tuned as desired for the specific drug product, wherein the “break” force may be high enough force to prevent expelling the drug product too quickly, but low enough so that it may not be difficult for the user to initiate the injection.

In some embodiments, device 80 may include a feedback mechanism. Device 80 may include a visual feedback mechanism, e.g., plunger rod 76 fully depressed into housing 74 (FIG. 7B) and needle cover 84 fully extended distally to housing 74 with needle 96 covered. In some examples, device 80 may include an opening for viewing container 92 and movement of plunger rod 76 and stopper 90 in container 92. Device 80 may also include an audible feedback mechanism, e.g., an audible “clicking” sound once a proximal end of plunger rod 76 abuts housing 74, or once needle cover 84 is pushed distally from housing 74. In some aspects, device 80 may include a tactile feedback, e.g., a snap or vibration as needle cover 84 moves distally and abuts ledge 98 at the end of the injection.

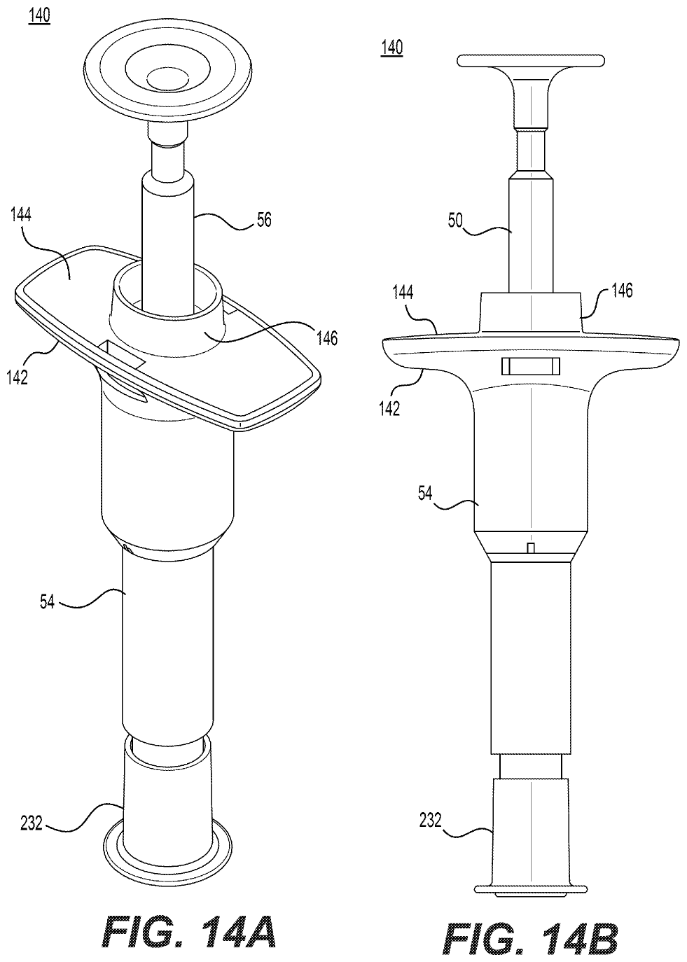

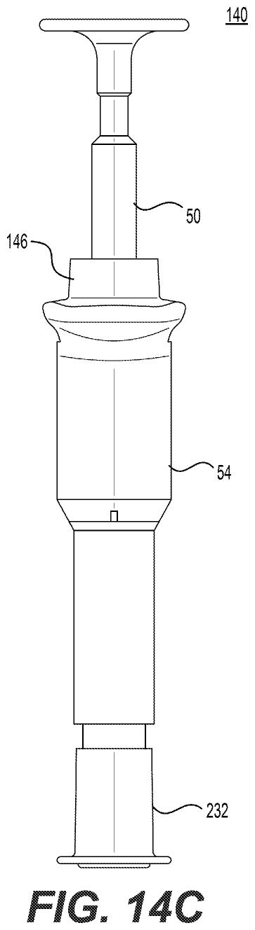

FIGS. 14A-14C depict another embodiment of the present disclosure, similar to those described above in reference to FIGS. 3A-7F. Where appropriate, features will be designated with references similar to those discussed above. Any features and components that correspond to those in FIGS. 3A-7F may be understood to be configured similarly. FIG. 14A shows a perspective view of the exterior of a device 140, FIG. 14B shows an exterior frontal view of device 140, and FIG. 14C shows an exterior side view of device 140. Device 140 may include plunger rod 56, housing 54, and needle cap 148. Housing 54 may include a finger flange 142 and rear cap 144. Finger flange 142 may have any appropriate size and/or configuration allowing for proper use. For example, a user may utilize two hands to operate device 140. One hand of the user may hold device 140, while the other hand of the user pushes plunger rod 56 in a distal direction towards finger flange 142. As shown in FIGS. 14A-14C, rear cap 144 may include a collar 146. Collar 146 may extend in a proximal direction away from finger flange 142. Collar 146 may be any appropriate size and/or configuration to surround a portion of plunger rod 56. In some embodiments, collar 146 may have a generally circular shape.

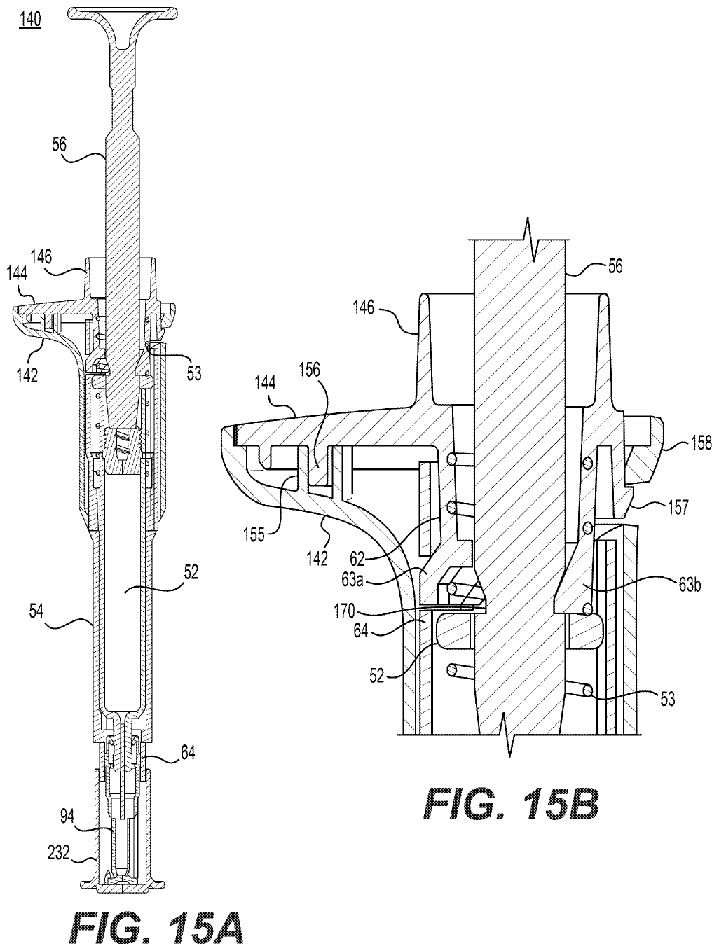

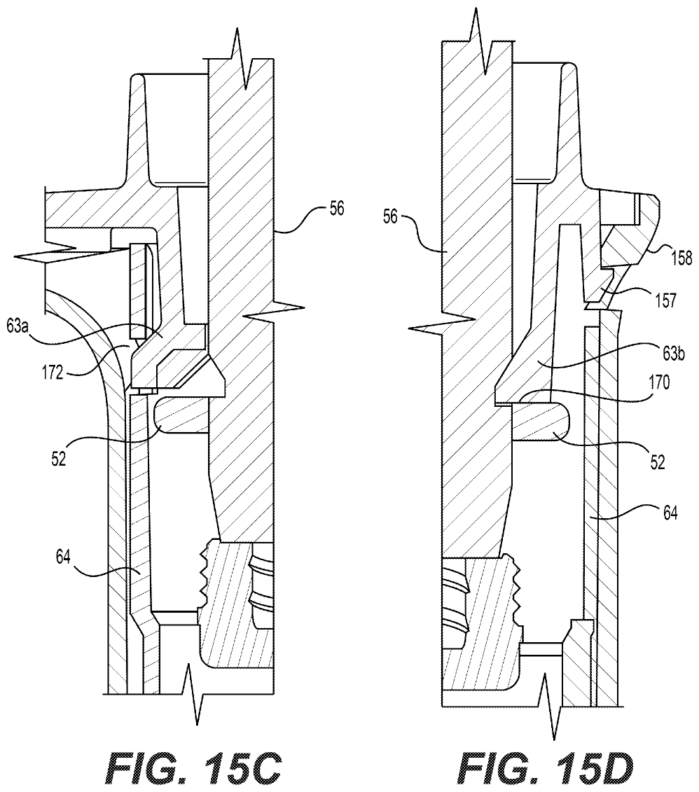

FIGS. 15A-15D depict a cross-sectional view of an interior of device 140. FIGS. 15A and 15B depict a cross-sectional view of a quarter portion of device 140. Referring to FIG. 15A, device 140 may include needle cover 64, syringe 52, spring shroud 53, needle cap 232, and needle shield 94. The configuration of needle cap 232 and needle shield 94 will be discussed in detail herein, e.g., in regards to FIGS. 22A and 22B. Referring to FIG. 15B, device 50 may include various features to couple finger flange 142 and rear cap 144 together. For example, finger flange 142 may include a snap 155 and rear cap 144 may include an insert 156. Snap 155 and insert 156 may engage, i.e., snap together, to couple rear cap 144 to finger flange 142. Rear cap 144 may also include a rear cap hook 157 for abutting against a ledge portion 158 of housing 14.

Referring to FIGS. 15B-15D, device 50 is shown in a pre-injection state. Rear cap 144 may include a blocking component 160. Blocking component 160 may correspond to blocking component 62 described above. Plunger rod 56 may be include an indent 170 and may be slidably coupled to blocking component 62, such that plunger rod 56 may slide in both a distal and proximal direction. Blocking component 62 may include deflectable arm 63 extending in a distal direction. As shown in FIG. 15B, blocking component 62 may include more than one deflectable arm 63. For example, blocking component 62 may include two, three, four, or more deflectable arms 63. In configurations with more than one deflectable arm 63, the deflectable arms 63 may be equally spaced apart from one another. For example, if the blocking component 62 includes four deflectable arms 63, each of the deflectable arms 63 may be about 90 degrees from one another.

In a pre-injection state, one or more of deflectable arms 63 may abut against a portion of plunger rod 56. For example, one or more of deflectable arms 63 may abut against indent 170 of plunger rod 56 (FIG. 15B). Needle cover 64 may include an opening 172 for receiving a portion of one or more of deflectable arms 63. In a pre-injection state, a first deflectable arm 63a may abut against plunger rod 56, such that a portion of first deflectable arm 63a may fit into opening 172 (FIG. 15C). A second deflectable arm 63 may abut against indent 170 (FIG. 15D). At this initial position, prior to use, significant motion of plunger rod 56 in either a proximal or a distal direction may be prevented. For example, if plunger rod 56 is pushed in a distal direction into housing 54, first deflectable arm 63a is prevented from deflecting outward by a distal end of needle cover 64, locking deflectable arm 63 within opening 172 and preventing further movement of plunger rod 56. If plunger rod 56 is pulled in a proximal direction away from housing 54, second deflectable arm 63b may be prevented from deflecting outward as second deflectable arm 63b abuts indent 170.

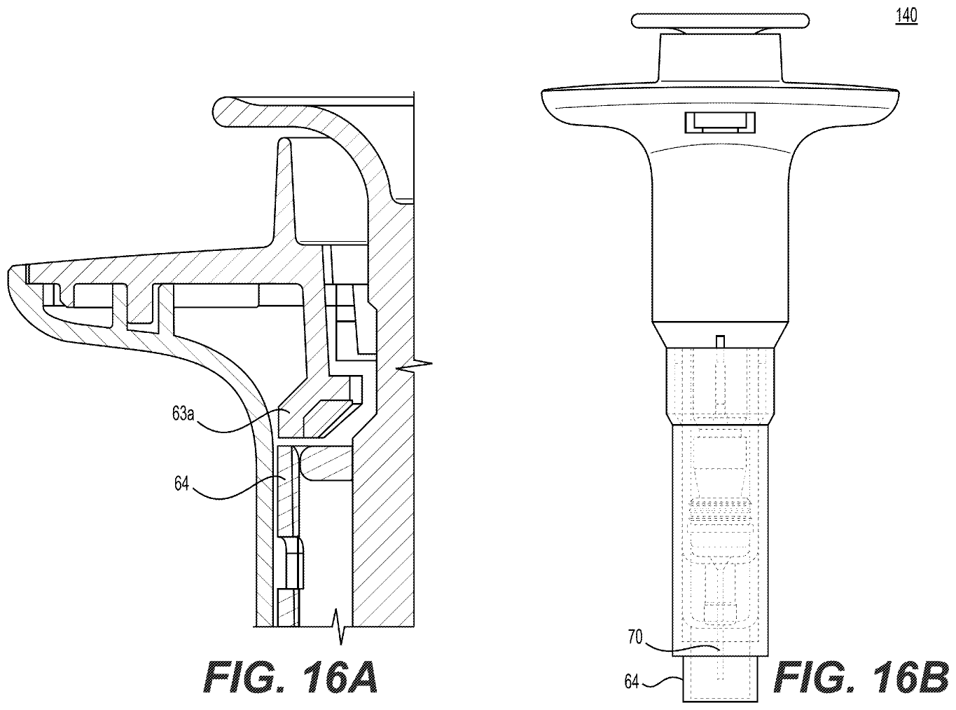

To initiate injection, device 140 may be pressed against an injection site to depress needle cover 64. As plunger rod 56 is pressed in a distal direction into housing 54, plunger rod 56 may deflect second deflectable arm 63b of blocking component 62, allowing plunger rod 56 to move distally into syringe 52 so that injection may proceed. Once injection is complete, device 140 may be removed from the injection site and needle cover 64 may automatically be pulled in a distal direction such that it cover the needle 70 (FIG. 16B). In some examples, spring shroud 53 may pull needle cover 64 into a distal direction. During injection, as plunger rod 56 moves distally into housing 54, first deflectable arm 63a may deflect inwards towards plunger rod 56, allowing first deflectable arm 63a to detach from opening 172. Referring to FIG. 16A, at the end of injection, and once first deflectable arm 63a is located proximally to needle cover 64, first deflectable arm 63a may return to an un-deflect position and needle cover 64 may be prevented from moving in a proximal direction. First deflectable arm 63a may now act as a lockout mechanism to prevent re-use of device 140 or accidental needle stick, since needle cover 64 may no longer be moved distally to expose needle 70. At this final, post-injection position of device 140, needle cover 64 may be in a lockout position and a proximal end of needle cover 64 and first deflectable arm 63a may abut one another. At this stage, device 140 may not be re-used.

Embodiments of device 140 depicted in FIGS. 14A-16B may include one or more additional features, e.g., optimal configuration and sizing of deflectable arms 62; ergonomic design to enable two hand operation, i.e., one hand may hold the syringe for needle insertion at 90 degrees, while the second hand pushes plunger rod 56; indicators of injection completion, e.g., a visual color change or audible indicator; activation points to mitigate potential failure modes, e.g., a pre-activation point or state; extension of plunger rod 56 to sit within a piston of the device to improve alignment of plunger rod 56 before and/or during use; and optimization of the design to reduce the required spring force to minimize plastic creep.

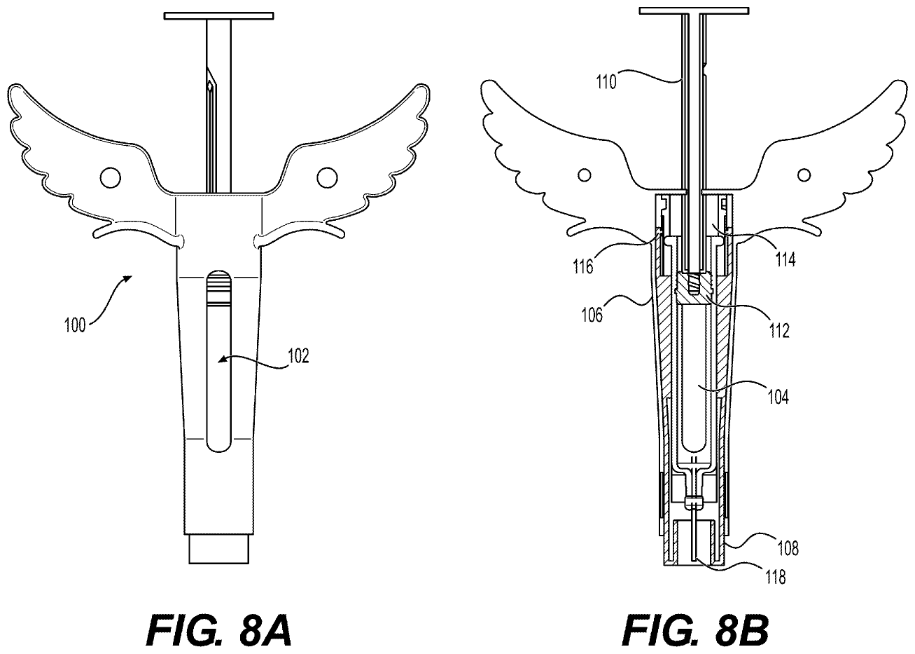

Another embodiment of the present disclosure is shown in FIGS. 8A-10B. FIG. 8A shows an exterior view of a device 100, while FIG. 8B shows a cross section of the device. Device 100 may be designed to hold any suitable commercially known product container 104 (e.g., a syringe or other suitable drug delivery device). Referring to FIG. 8B, device 100 may include a housing 106, needle cover 108, plunger rod 110, stopper 112, and rotating component 114, e.g., a cam.

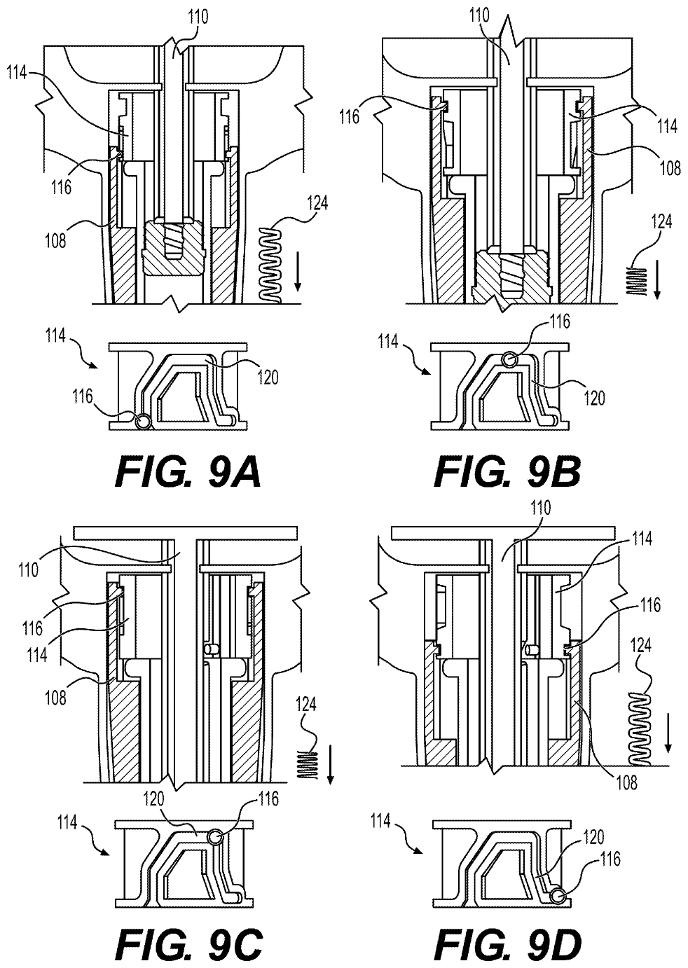

Prior to injection, needle cover 108 may extend distally from housing 106 to cover needle 118. To initiate injection, device 100 may be pressed against an injection site. Device 100 may include a spring (not shown), e.g., around or at an end of needle cover 108. An exemplary actuator, e.g., spring, 124 is shown schematically in FIGS. 9A-9D for discussion purposes. An upper region of each of FIGS. 9A-9D depicts a cross section of plunger rod 110, rotating component 114, and projection 116 of needle cover 108 that interacts with track 120 of rotating component 114. A bottom region of each of FIGS. 9A-9D depicts a side view of movement of projection 116 of needle cover 108 along track 120 of rotating component 114 that corresponds to the cross-sectional view of that figure.

As shown in FIGS. 9A-9D, rotating component 114 may include a track 120, i.e., a groove, for receiving a protrusion 116 of needle cover 108. At an initial position (FIG. 9A), actuator 124 may be uncompressed, needle cover 108 may extend distally over needle 118, and plunger rod 110 may be locked in position relative to rotating component 114. This may prevent plunger rod 110 from being pushed distally into housing 106 until needle cover 108 is pushed proximally to reveal needle 118. To initiate injection, plunger rod 110 may be twisted relative to rotating component 114, allowing plunger rod 110 to move from a locked position to an unlocked position. Needle cover 108 may be pushed proximally into housing 106, e.g., by a user pressing device 100 against an injection site, which may cause rotating component 114 to turn and actuator 124 to compress during injection (FIG. 9B). Plunger rod 110 may be locked in place, i.e., movement of plunger rod 110 may be prevented, until needle cover 108 is retracted proximally into housing 106. Such configuration may prevent premature expulsion of the product. During injection, needle cover 108 may be held in the retracted position due to the location of protrusion 116 on needle cover 108 in track 120 of rotating component 114 (FIG. 9B). This may act as a signal to the user that device 100 is still in use. In this retracted position, needle cover 108 may also control insertion depth of needle 118, since a distal end of needle cover 108 may prevent deeper insertion of needle 118 once the distal end of needle cover 108 contacts the injection site. Once injection is complete, plunger rod 110 may turn rotating component 114 to free needle cover 108 (FIG. 9C), and actuator 124 may push needle cover 108 back down to a lockout position, wherein needle cover 108 extends distally from housing 106 (FIG. 9D) to cover needle 118 once the injection is complete. As can be seen in FIG. 9D, in the lockout position, at the end of the injection, protrusion 116 on needle cover 108 may engage a notch at the end of track 120 on rotating component 114 that prevents needle cover 108 from being pushed proximally into housing 106 again to prevent re-use of device 100.

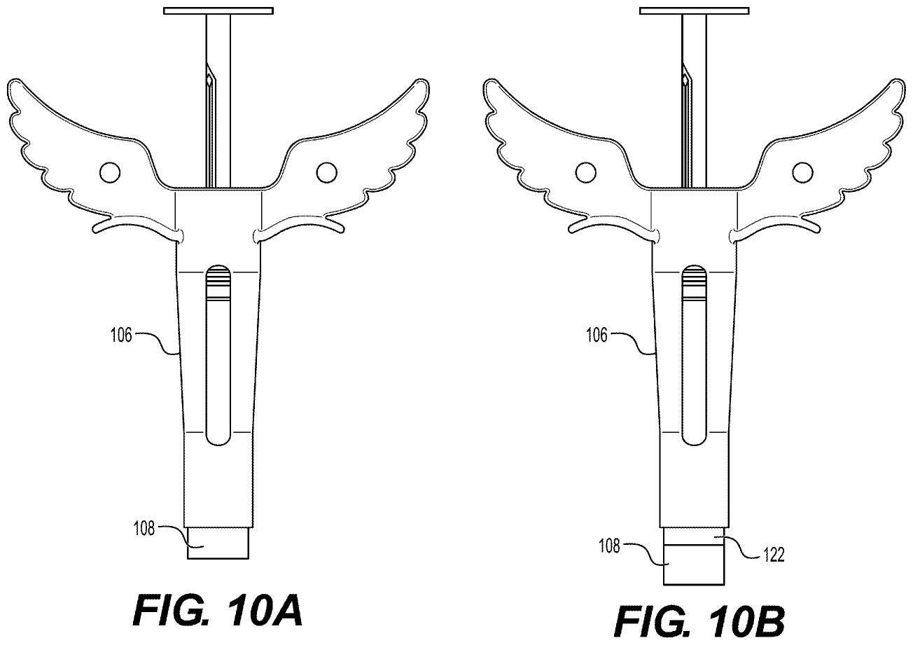

In some embodiments, in the lockout position, needle cover 108 may be pushed out farther distally than the initial position of needle cover 108. For example, FIG. 10A depicts an initial position of needle cover 108 prior to injection, and FIG. 10B depicts a lockout position of needle cover 108 at end-of-dose. Portion 122 represents an additional portion of needle cover 108 that may be exposed at the end of the injection, but not at the starting point of the injection. In some embodiments, the height of track 120 rotating component 114 may be greater at the end compared to the beginning of track 120. For example, the beginning of track 120 (shown at the left side of rotating component 114 in FIGS. 9A-9D) may start at a central region of rotating component 114, may continue to an upper region of rotating component 114, and then the end of track 120 (shown at the right side of rotating component 114 in FIGS. 9A-9D) may be located at a lower region of rotating component 114. Accordingly, the end of track 120 may have a height greater than a height of the initial part of track 120. This increased height at the end of track 120 may allow needle cover 108 to extend out further at its lockout position than at its original position (FIG. 10B).

The embodiments of FIGS. 8A-10B depict a device with a passive safety mechanism that may activate when the user nears end-of-dose, i.e., when a substantial amount of the product is fully expelled from container 104. The passive safety mechanism of FIGS. 8A-10B may allow for plunger rod 110 to turn rotating component 114 when end-of-dose is approach, which may push needle cover 108 proximally to an extended lockout position. In the lockout position, needle cover 108 may not retract back into housing 106. In such a lockout position, as well as prior to injection, needle cover 108 may cover needle 118 to prevent accidental needle stick post-injection. This may also reduce needle-phobia and needle-related anxiety as compared to conventional drug delivery devices with exposed needles. In addition, needle cover 108 may allow for controlled needle insertion depth. In other words, exposing only a portion of needle 118 during injection may allow for usage of device 100 without any pre-injection steps, e.g., pinching the skin to alter the injection depth to compensation for a needle length that is longer compared to the exposed needle 118 of device 100. Device 100 may be configured, e.g., container 104 may be positioned within housing 106 and/or needle cover 108 may be sized, so that needle cover 108 exposes only a portion of needle 118, and the length of the exposed portion of needle 118 represents the desired insertion depth of needle 118. As discussed above, during injection, needle cover 108 may be held back in a retracted position (FIG. 9B) due to the position of protrusion 116 on needle cover 108 in track 120 of rotating component 114. This may prevent the passive safety mechanism from activating prior to end-of-dose. Additionally, if device 100 is removed from the injection, accidentally or on purpose, passive safety mechanism may not activate, as needle cover 108 is held in track 120. Once device 100 is reapplied to the injection site and plunger rod 110 may be depressed, injection may resume.

In some embodiments, device 100 may include a feedback mechanism. Device 100 may include a visual feedback mechanism, e.g., plunger rod 110 abutting housing 106, needle cover 108 fully extended distally from housing 106, and/or an extended portion 122 distal of housing 106 (FIG. 10B). In some examples, needle cover 108 may have a first color, pattern, or texture, e.g., and extended portion 122 may have a second color, pattern, or texture, to show that device 100 is in the lockout position, and the device has been used and the dose has been expelled. In some examples, device 100 may include an opening 102 for viewing container 104 and movement of plunger rod 110 and/or stopper 112 in container 104. Device 100 may also include an audible feedback mechanism, e.g., an audible “clicking” sound once plunger rod 110 abuts housing 106 and/or needle cover 108 moves distally at the end of the dose. In some aspects, device 100 may include a tactile feedback, e.g., a snap or vibration as needle cover 108 moves distally at the end of the injection.

Device 100 may be of any suitable size and shape to hold or partially hold a container 104, and/or to support and hold any components described herein. Any components of device 100 may be of a suitable size and/or shape to configure to and cooperate with other components as designed. For example, rotating component 114 may be of any suitable size and/or shape to slidably couple to plunger rod 110. In another example, needle cover 108 and/or protrusion 116 may be of any suitable size and/or shape to fit into track 120 of rotating component 114.

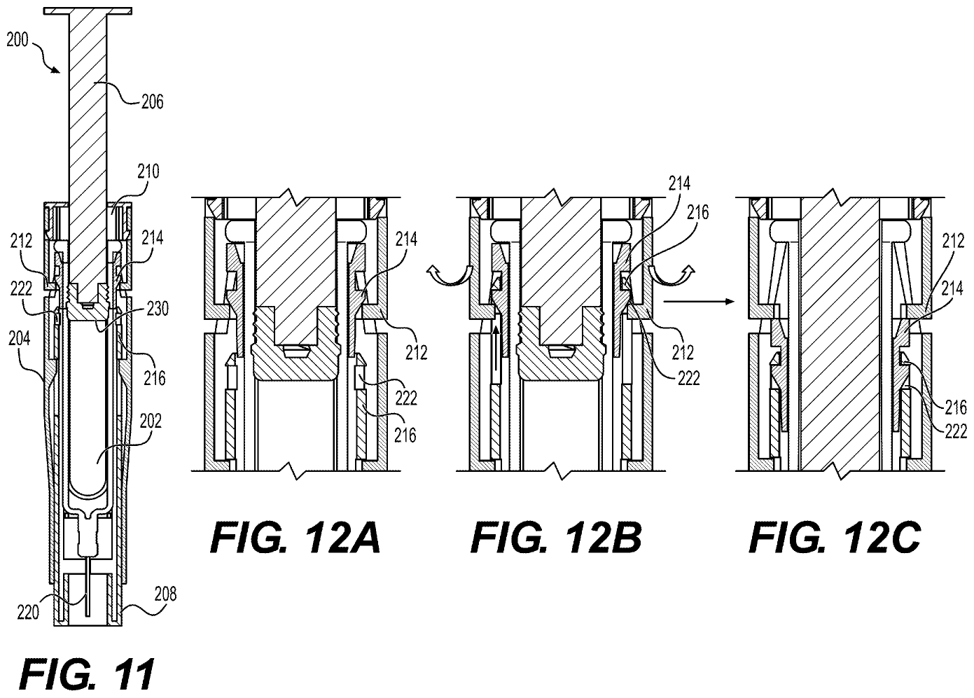

Another embodiment of the present disclosure is shown in FIGS. 11-13B. FIG. 11 shows an exterior view of a device 200. Device 200 may be designed to hold any suitable commercial product container 202 (e.g., a syringe or other suitable drug delivery device). Referring to FIG. 11, device 200 may include a housing 204, plunger rod 206, needle cover 208, and stopper 230. Proximal region of housing 204 may comprise one or more deflectable housing tabs 212. Housing 204 may also include a cap 210 located within a proximal portion of housing 204. Device 200 may include a lockout ring 214 slidably coupled to a proximal end of container 202. In some embodiments, housing 204 may include more than one deflectable housing tabs 212. For example, housing 204 may include two, three, four, or more deflectable housing tabs 212. In configurations with more than one deflectable housing tabs 212, the deflectable housing tabs 212 may be equally spaced apart from one another. For example, if the housing 204 includes four deflectable housing tabs 212, each of the deflectable housing tabs 212 may be about 90 degrees from one another. If the housing 204 includes two deflectable housing tabs 212, each of the deflectable housing tabs 212 may be about 180 degrees from one another.

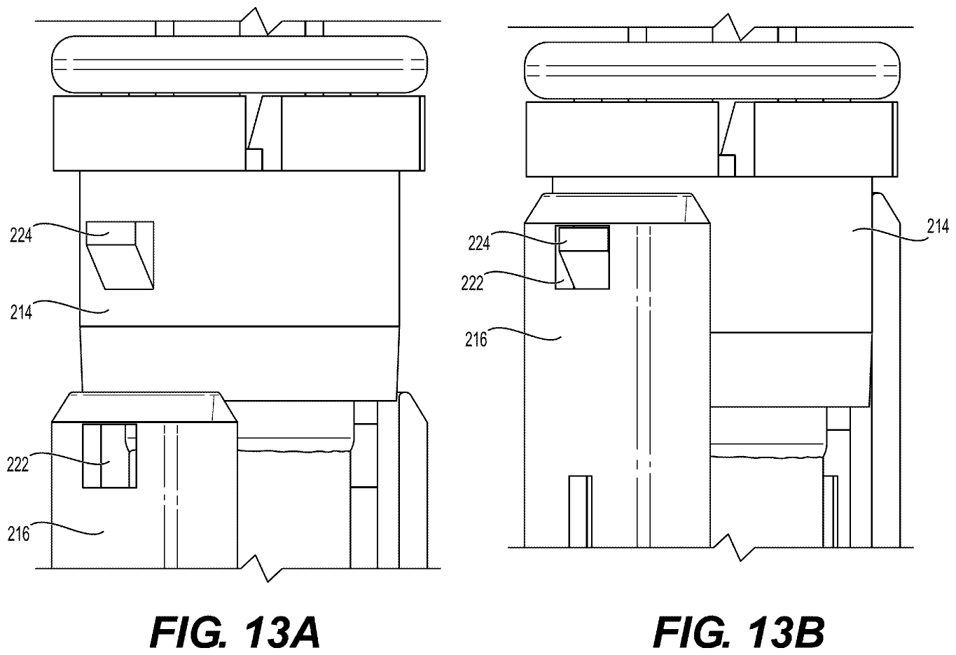

As shown in FIG. 11, needle cover 208 may be extended and covering needle 220 prior to use. At an initial position (FIG. 12A), deflectable housing tab 212 may abut a portion of lockout ring 214, and lockout ring 214 may be located proximally to a proximal end 216 of needle cover 208. The needle cover may be biased, e.g., by a spring, in the distal direction, but may be pushed back proximally within housing 204 for use. To initiate injection, needle cover 208 may be pushed in a proximal direction into housing 204, e.g., by pushing device 200 against an injection site. When needle cover 208 is moved proximally, proximal end 216 of needle cover 208 may hook onto a ledge 224 of lockout ring 214 (FIG. 12B). Movement of proximal end 216 of needle cover 208 relative to lockout ring 214 is shown in further detail in FIGS. 13A-13B. In some examples, proximal end 216 may include a cutout 222 for engaging with ledge 224. In some examples, needle cover 208 may include more than one cutout 222, e.g., a pair of cutouts, and lockout ring 214 may include more than one ledge 224, e.g., a pair of ledges. The number of ledges and cutouts may correspond with one another, so that each ledge may engage with each cutout. Although lockout ring 214 is depicted as including one or more ledges 224, and proximal end 216 of needle cover 208 is depicted as including one or more cutouts 222, the two mating regions may be reversed, i.e., lockout ring 214 may include one or more cutouts 222, and needle cover 208 may include one or more ledges 224. Further, ledges 224 and cutouts 222 may represent any suitable mating portions.

With needle cover 208 pushed proximally into housing 204, e.g., by pressing device 200 against an injection site, plunger rod 206 may be pushed in a distal direction into housing 204 to expel a product from container 202 into the injection site.

The movement of proximal end 216 of needle cover 108 into housing 204 may flex deflectable tab 212 outwards away from plunger rod 206 during injection, as shown by the arrows pointing radially outwards in FIG. 12B. Once device 200 is removed from the injection site, e.g., when injection is complete, needle cover 208, along with lockout ring 214, may be biased to retreat back downwards to its initial position, e.g., via a spring force of a spring shroud (not shown) around needle cover 208. Needle cover 208 and lockout ring 214, connected to one another via cutouts 222 and ledges 224, may move downwards past deflectable tab 212, allowing deflectable tab 212 to flex back to its initial position (FIG. 12C). As shown in FIG. 12C, a proximal end of lockout ring 214 may abut a distal end of deflectable tab 212. At this final, end of dose position, needle cover 208 may be in a lockout position, where needle cover 208 may be prevented from moving proximally back into housing 204 by deflectable tab 212, which may obstruct its path.

The embodiments of FIGS. 11-13B depict a device with a passive safety mechanism that may activate at any point when device 200 is removed from the injection site, whether or not the entire dose has been expelled from container 202. Once needle cover 208 has been pushed proximally into housing 204 to initiate the injection, the passive safety mechanism may be activated. Since needle cover 208 is biased in the distal direction, once the force pushing needle cover 208 into housing 204 is removed (e.g., by removal of device 200 from a user's skin), the safety mechanism may be activated. Such a passive safety mechanism may be helpful for users who may be concerned about needle safety. For example, if a user accidentally drops device 200 during use, the passive safety mechanism may push needle cover 208 out to prevent accidental needle stick. Additionally, needle 220 may be covered by needle cover 208 and hidden from view prior to, during, and post injection. This may reduce needle-phobia and needle-related anxiety as compared to conventional drug delivery devices with exposed needles. In addition, needle cover 108 may allow for controlled needle insertion depth. In other words, exposing only a portion of needle 220 during injection may allow for usage of device 200 without any pre-injection steps, e.g., pinching the skin to alter the injection depth to compensate for a needle length that is longer compared to the exposed needle 220 of device 200. Device 200 may be configured, e.g., container 202 may be positioned within housing 204 and/or needle cover 208 may be sized, so that needle cover 208 exposes only a portion of needle 220, and the length of the exposed portion of needle 220 represents the desired insertion depth of needle 220.

In some embodiments, device 200 may include a feedback mechanism. Device 200 may include a visual feedback mechanism, e.g., plunger rod 206 abutting housing 204, needle cover 208 fully extended distally from housing 204. In some examples, device 100 may include an opening for viewing container 202 and movement of plunger rod 206 and/or stopper 230 in container 202. Device 200 may also include an audible feedback mechanism, e.g., an audible “clicking” sound once plunger rod 206 abuts housing 204, when needle cover 208 extends distally, and/or when deflectable tab 212 relaxes back to its initial, neutral position (FIG. 12C). In some aspects, device 200 may include a tactile feedback, e.g., a snap or vibration as needle cover 208 moves distally.

Device 200 may be of any suitable size and shape to hold or partially hold a product container 202, and/or to support and hold any components described herein. Any components of device 200 may be of a suitable size and/or shape and configured to cooperate with other components as designed. For example, needle cover 208, proximal end 216, cutout 222, lockout ring 214, and ledge 224, may be of any suitable size and/or shape allowing for cutout 222 to lock onto ledge 224.

FIGS. 22A and 22B depict embodiments of needle shield 94 and needle cap 232 that may be used with any of the devices disclosed herein. Needle shield 94 may attach to a needle cover (not shown) and may extend in a distal direction to fully enclose needle 96. As shown in FIGS. 22A and 22B, needle shield 94 may extend in a distal direction such that needle shield 94 may abut a portion of needle cap 232. Needle cap 232 may include a clip 221. In other embodiments, needle cap 232 may include a plurality of clips 221. Clip 221 may be shaped or configured to attach to, i.e., latch onto, a ledge 223 of needle shield 94. Needle cap 232 as depicted in FIGS. 22A and 22B show an arm 95 of needle shield 94. In other embodiments, needle shield 94 may include a plurality of arms 95 and a gap between each arm 95. In some examples, needle cap 232 may include at least two clips, at least three clips, or at least four clips. Referring to FIG. 22B, a first clip 221a may attach onto a first ledge 223a and abut a first arm 95 of needle shield 94. A second clip 221b may attach to a second ledge 223b. As the user pulls off needle cap 232 in a distal direction away from housing 14, needle shield 94 may also simultaneously out of housing 14, to expose needle 86.

Additional concepts and features that may be used with or in any of the devices disclosed herein will be discussed below. The following concepts and features may correspond to concepts and features as discussed throughout the present disclosure.

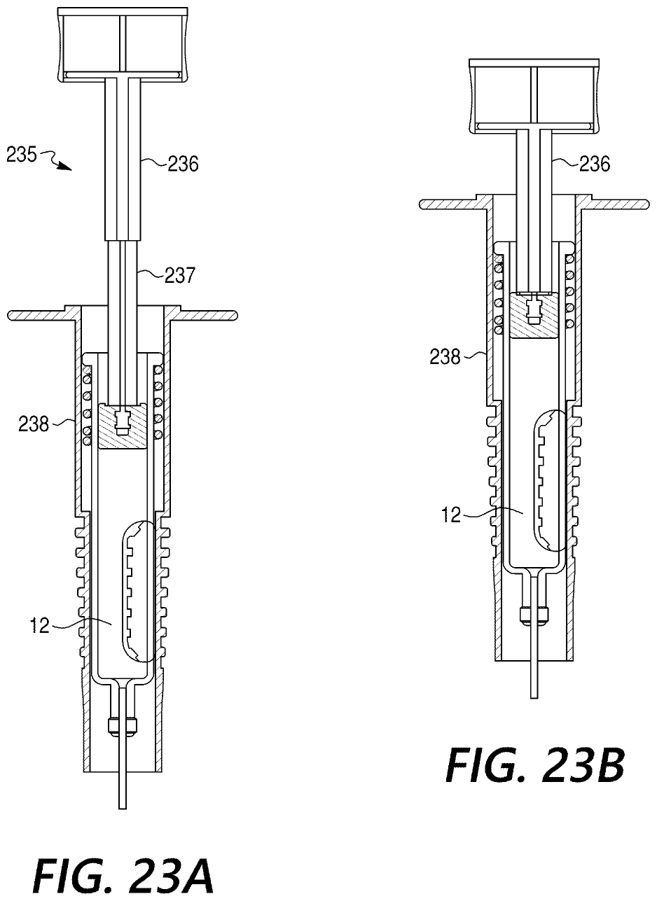

FIGS. 23A and 23B depict a plunger rod 235 that may be utilized in any of the embodiments discussed herein. Plunger rod 235 may include a first portion 236 and a second portion 237. As shown in FIG. 23A, second portion 237 may have a width that is less than a width of first portion 236. Referring to FIG. 23B, as plunger rod 235 moves distally toward housing 238, dispensing a medicament out of syringe 12, second portion 237 may collapse into, i.e., insert into, first portion 236. In other embodiments, first portion 236 may have a width that is less than width of second portion 237, such that as plunger rod 235 moves distally toward housing 238, first portion 236 may collapse into second portion 237. Plunger rod 235 may be referred to as a telescoping plunger rod. As shown in FIGS. 23A and 23B, as plunger rod 235 collapses, a length of plunger rod 235 decreases from a first length (FIG. 23A) to a second length (23B).

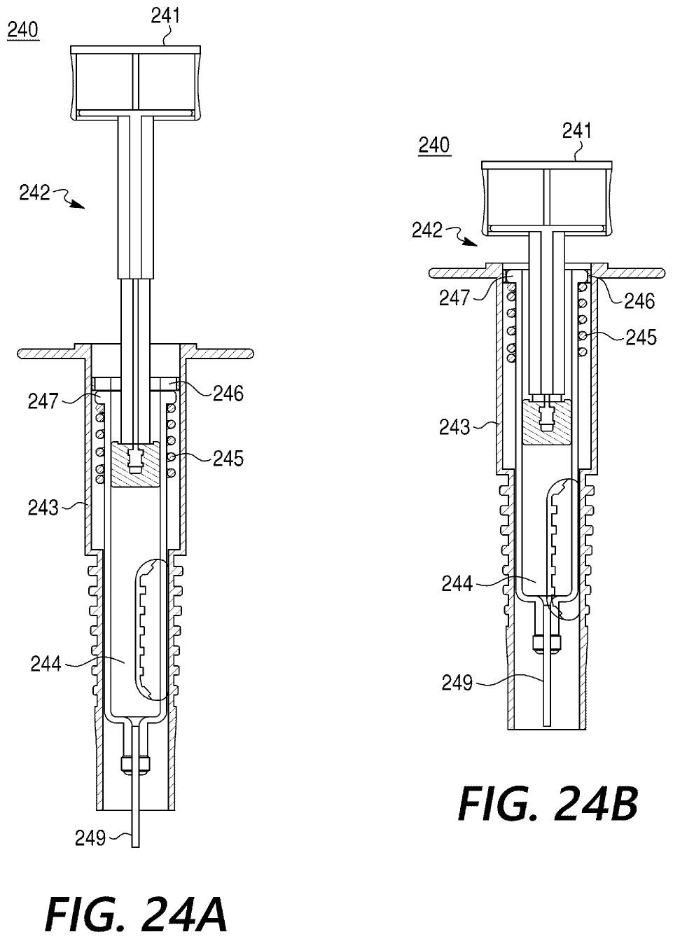

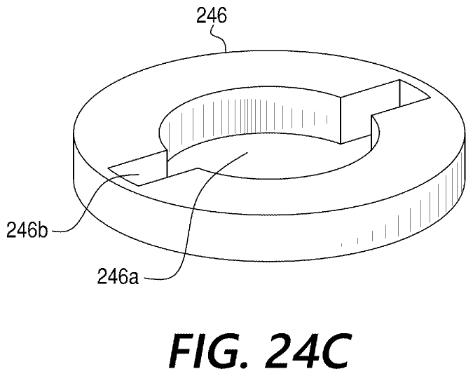

FIGS. 24A-24C depict an embodiment of the present disclosure. Device 240 may include a thumb pad 241, a plunger rod 242, a housing 243, a syringe 244, a spring shroud 245, a rotator 246, and a needle 249. In a pre-injection state, as depicted in FIG. 24A, rotator 246 may be located proximally to a proximal end of syringe 244. Referring to FIG. 24C, rotator 246 may include an opening 246a shaped and configured to surround a portion of plunger rod 242. In some examples, opening 246a may have a circular shape such that a portion of plunger rod 242 may slide in a distal direction through rotator 246. Rotator 246 may also include a plurality of cutouts 246b. Cutouts 246b may have a shape, size, and configuration, corresponding to a proximal end of syringe 244. For example, cutouts 246b may correspond to a flange 247 of syringe 244. In a pre-injection state, rotator 246 may be located proximally to a proximal end of syringe 12 such that cutouts 246b are displaced from flange 247. Spring shroud 245 may surround a proximal end of syringe 244, such that syringe 244 may be spring loaded and held down by rotator 246 (FIG. 24A). To initiate injection, a user may depress thumb pad 241 in a distal direction towards housing 243. During injection, as plunger rod 242 moves in a distal direction into housing 243 and syringe 244, plunger rod 242 may rotate such that cutouts 246b rotate towards flange 247. At end-of-dose, as depicted in FIG. 24B, syringe 244 may move in a proximal direction into housing 243 and into rotator 246, and needle 249 may be contained within a distal end of housing 243. In some embodiments, rotator 246 and syringe 244 may not be coupled together and then may move proximally towards a proximal end of housing 243. Any appropriate syringe 244 may be used with device 240. For example, syringe 244 may be a cyclo-olefin-polymer syringe or a glass cut flange syringe.

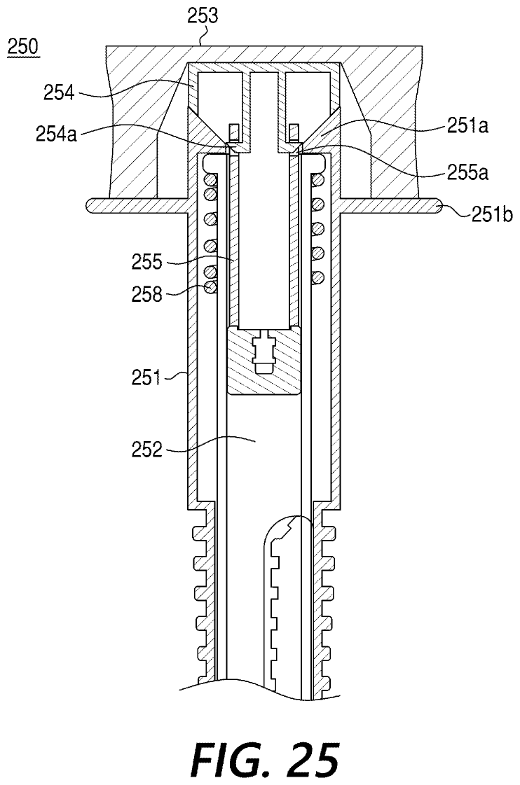

FIG. 25 depicts an embodiment of the present disclosure. Device 250 may include a housing 251, a syringe 252, and a thumb pad 253. Device 250 may also include a telescoping plunger rod including a first portion 254 and a second portion 255. The telescoping plunger rod may correspond to plunger rod 235 depicted in FIGS. 23A and 23B. As shown in FIG. 25, first portion 254 may include plurality of ledges 254a and second portion 255 may include a plurality of cutouts 255a. Ledges 254a may fit into cutouts 255a, coupling first portion 254 to second portion 255. At end-of-dose, thumb pad 253 may abut a flange portion 251b of housing 251. Housing 251 may include proximal extensions 251a. Referring to FIG. 25, at end-of-dose, once thumb pad 253 abuts flange portion 251b, first portion 254 and second portion 255 may also abut proximal extensions 251a. As first portion 254 and second portion 255 abut proximal extensions 251a, ledges 254a may push against proximal extensions 251a, deflecting proximal extensions 251a outwards and away from syringe 252. Spring shroud 258 may then draw syringe 252 and/or second portion 254 proximally into thumb pad 253, drawing a needle at the distal end of the syringe (not shown) into housing 251. In some embodiments, thumb pad 253 and housing 251 may include attachment features for locking thumb pad 253 and housing 251 together at the end-of-dose stage.

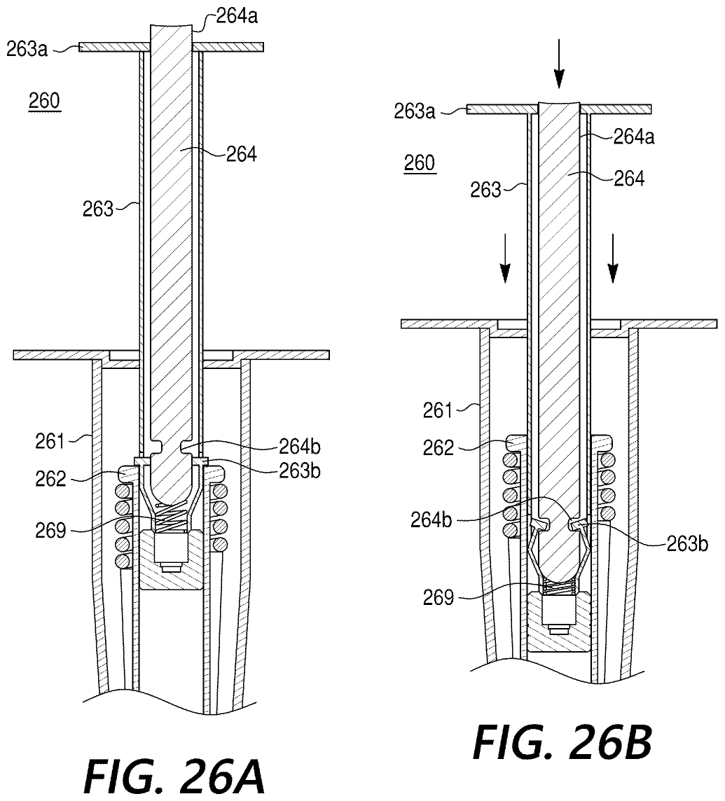

FIGS. 26A and 26B depict an embodiment of the present disclosure. Device 260 may include a housing 261, a syringe 262, and a plunger rod 263. Device 260 may also include an internal rod 264 extending through plunger rod 263. As shown in FIG. 26A, internal rod 264 may include a button 264a extending proximally from a thumb pad 263a of plunger rod 263. In a pre-injection state, deflectable arms 263b of plunger rod 263 may abut against a distal portion of internal rod 264, preventing movement of plunger rod 263 and as such, preventing pre-expulsion of the contents of syringe 262. A spring 269 may be included in plunger rod 263. To initiate injection, a user may press button 264a while simultaneously pushing thumb pad 263a in a distal direction towards housing 261 and against spring 269. Pressing button 264a may move internal rod 264 in a distal direction towards housing 261. Referring to FIG. 26B, as internal rod 264 moves distally into housing 261, deflectable arms 263b may then deflect inwards into indents 264b. Once deflectable arms 263b are deflected into indents 264b, plunger rod 263 may freely move in a distal direction to dispense the contents of syringe 262.

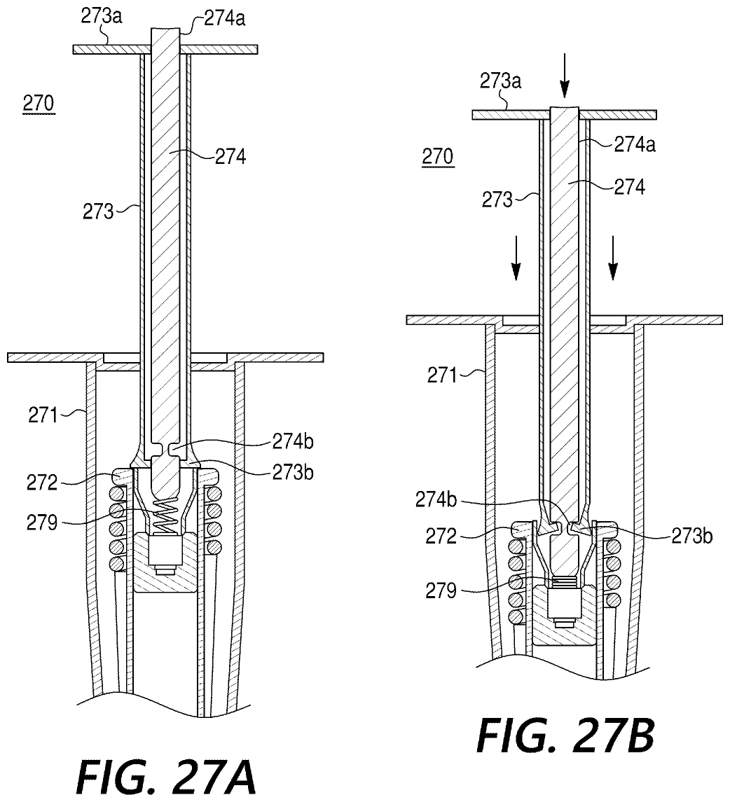

FIGS. 27A and 27B depict a device 270 similar to device 260 in FIGS. 26A and 26B. Device 270 may include a housing 271, a syringe 272, and a plunger rod 273. Device 270 may also include an internal rod 274. As shown in FIG. 27A, internal rod 274 may include a button 274a extending proximally from a thumb pad 273a of plunger rod 273. In a pre-injection state, deflectable arms 273b of plunger rod 273 may abut against a distal portion of internal rod 274, preventing movement of plunger rod 273 and as such, preventing pre-expulsion of the contents of syringe 272. A spring 279 may be included in plunger rod 273. To initiate injection, a user may press button 274a while simultaneously pushing thumb pad 273a in a distal direction towards housing 271 and against spring 279. Pressing button 274a may move internal rod 274 in a distal direction towards housing 271. Referring to FIG. 27B, as internal rod 274 moves distally into housing 271, deflectable arms 273b may then deflect inwards into indents 274b. Once deflectable arms 273b are deflected into indents 274b, plunger rod 273 may freely move in a distal direction to dispense the contents of syringe 272.

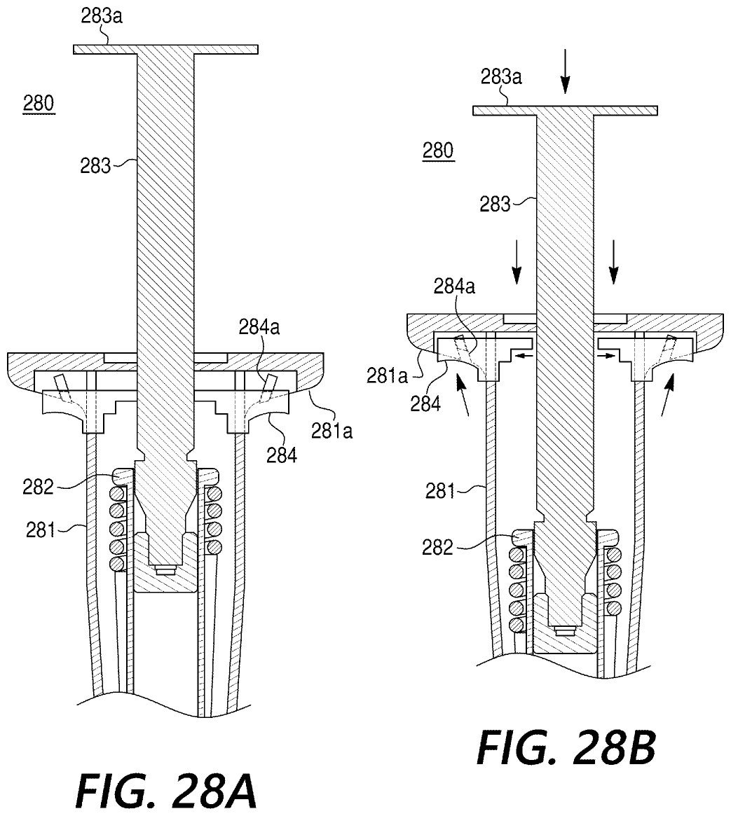

FIGS. 28A and 28B depict another embodiment according to the present disclosure. Device 280 may include a housing 281, a syringe 282, and a plunger rod 283. As shown in FIGS. 28A and 28B, housing 281 may include a flange 281a. Housing 281 may also include a trigger 284. FIGS. 28A and 28B show an interior view of device 280. Trigger 284 may extend through an internal portion of housing 281, for example, flange 281a. Referring to FIG. 28A, a portion of trigger 284 may extend distally from flange 281a. Flange 281a may include a plurality of slots 284a. Slots 284a may move trigger 284 within flange 281a. In a pre-injection state, as shown in FIG. 28A, a portion of trigger 284 abuts against plunger rod 283, preventing pre-expulsion of the contents of syringe 282. To initiate injection, a user may push thumb pad 283a distally towards housing 281, while simultaneously squeezing a portion of trigger 284 that extends distally from flange 281a. For example, the user may squeeze the portion of trigger 284 in a proximal direction into flange 281a. As the portion of trigger 284 is squeezed, slots 284a may guide trigger 284 in a direction away from plunger rod 283, allowing plunger rod 283 to move in a distal direction into housing 281 (FIG. 28B).

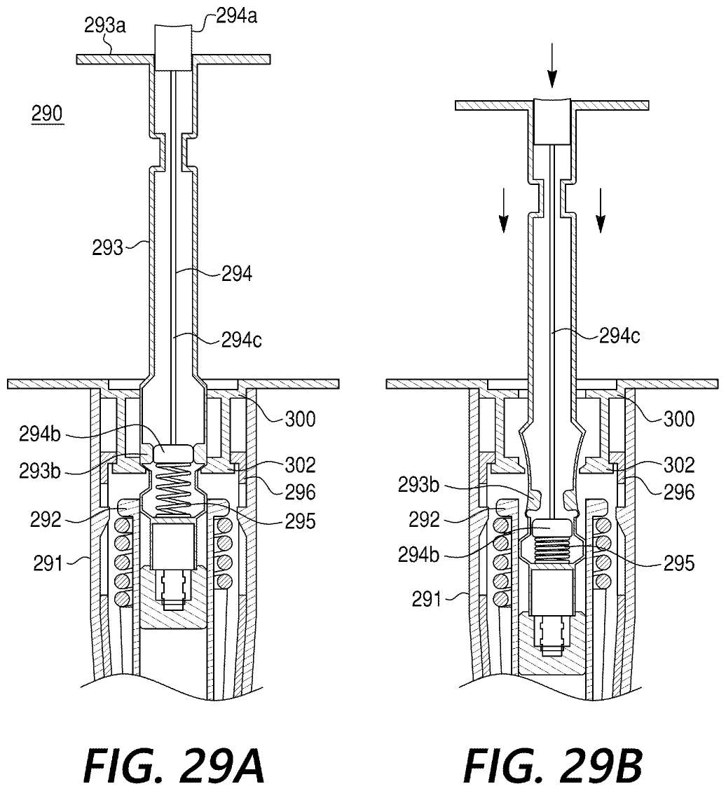

FIGS. 29A and 29B depict another embodiment according to the present disclosure. Device 290 may include a housing 291, syringe 292, plunger rod 293, and internal rod 294. As shown in FIG. 29A, device 290 may include a blocking component 300. Blocking component 300 may correspond to blocking components 62 and 82 as described herein. Blocking component 300 may include deflectable arms 302. Deflectable arms 302 may correspond to deflectable arms 63 and 83 as described herein. Internal rod 294 may include a button 294a extending proximally from thumb pad 293a of plunger rod 293. Internal rod 294 may also include a distal block 294b. In a pre-injection state, as shown in FIG. 29A, distal block 294b may be positioned above a spring 295 and within a flexible portion 293b of plunger rod 293. Distal block 294b may abut against flexible portion 293b, preventing movement of deflectable arms 302. This pre-injection state may prevent pre-expulsion of contents from syringe 292. To initiate injection, a user may press button 294a distally into plunger rod 293. Pressing button 294a may move internal rod 294 and distal block 294b in a distal direction towards syringe 292 and against spring 295. As shown in FIG. 29B, distal block 294b may be moved in a distal direction away from flexible portion 293b of plunger rod 293. Deflectable arms 302 may then deflect inwards towards flexible portion 293b, which may then allow plunger rod 293 to move distally into housing 291 and syringe 292 to expel the contents of syringe 292.

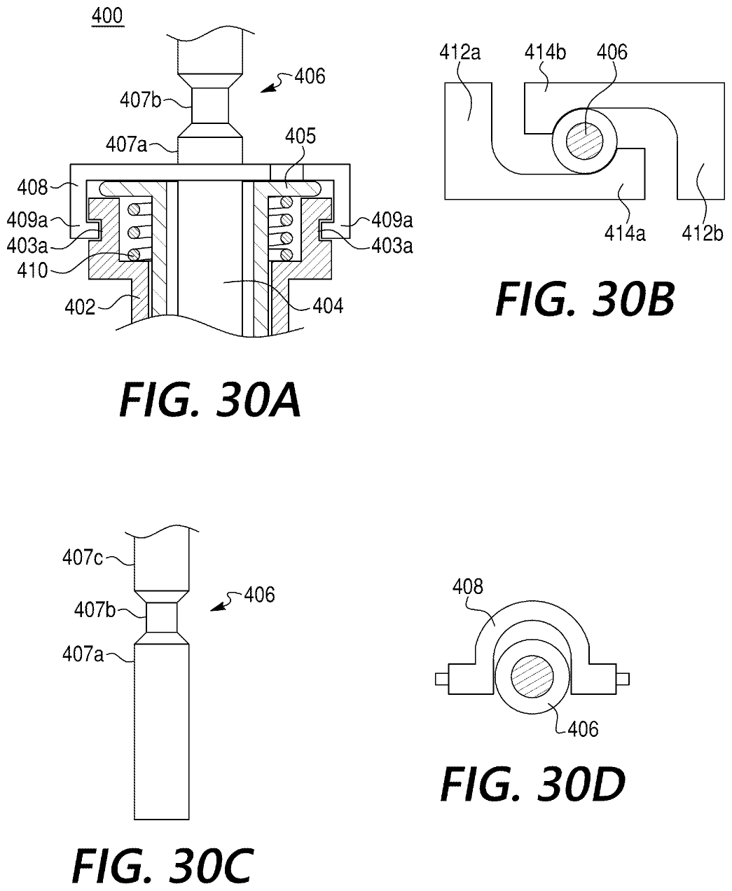

FIGS. 30A-30 depict another embodiment according to the present disclosure. Device 400 may include a housing 402, a syringe 404, a plunger rod 406, a rear cap 408, and a spring 410 surrounding a proximal portion of syringe 404. As shown in FIG. 30A, a proximal portion of housing 402 may include a plurality of recesses 403a, 403b and rear cap 408 may include a plurality of distal arms 409a, 409b. Recesses 403, 403b may be configured to receive distal arms 409a, 409b. To couple rear cap 408 to housing 402, a first distal arm 409a may hook into recess 403a and a second distal arm 409b may hook into recess 403b. Syringe 404 may also include a syringe flange 405. As shown in FIG. 30A, syringe flange 405 may sit between a portion of finger flange 408 and a portion of housing 402. For example, a proximal end of syringe flange 405 may abut a distal interior surface of finger flange 405 and a distal end of syringe flange 405 may abut a proximal end of housing 402. Referring to FIGS. 30A and 30C, plunger rod 406 may include a first portion 407a and a second portion 407b. First portion 407a may have a first diameter and second portion 407b may have a second diameter. The first diameter may be larger than the second diameter. In other examples, the first diameter may be smaller than the second diameter. In some examples, the plunger rod may have a first width and a second width, wherein the first width may be larger than the second width. In other examples, the first width may be smaller than the second width. As shown in FIG. 30C, plunger rod 406 may include a third portion 407c, wherein a diameter of third portion 407c is larger than the diameter of second portion 407b. Finger flange 408 may be any appropriate size, shape, and/or configuration to surround a portion of plunger rod 406.

Referring to FIG. 30B, finger flange 408 may comprise a first component 412a and a second component 412b. First component 412a may include a first hook 414a and second component 412b may include a second hook 414b. As shown in FIG. 30B, first hook 414a and second hook 414b may abut and/or surround a portion of plunger rod 406. At a pre-injection state, as shown in FIGS. 30A and 30B, finger flange 408 may surround first portion 407a of plunger rod 406. Finger flange 408 may also include a spring (not shown) configured to separate first component 412a and second component 412b (FIG. 30B). Movement of finger flange 408 may be prohibited by the spring and engagement of first hook 414a and second hook 414b with first portion 407a of plunger rod 406. To initiate injection, a user may depress a thumb pad (not shown) plunger rod 406 in a distal direction towards housing 402. During injection, plunger rod 406 may be located in housing 402 such that second portion 407b of plunger rod 406 is received in finger flange 408. In embodiments where second portion 407b may have a diameter that is less than a diameter of first portion 407a, hooks 414a, 414b may no longer abut a portion of plunger rod 406. For example, as shown in FIG. 30D, there may be a space between finger flange 408 may and plunger rod 406, allowing plunger rod 406 to move distally into housing 402 and syringe 404 to dispel contents out of syringe 404. At end-of-dose, as shown in FIG. 30A, the user may maintain depression of the thumb pad, allowing a portion of plunger rod 406 to retreat backwards, i.e., proximally away from housing 402. Spring 410 may then be released, allowing syringe 404 to move proximally towards a distal surface of finger flange 408.

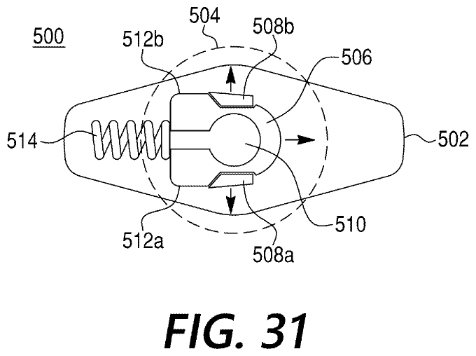

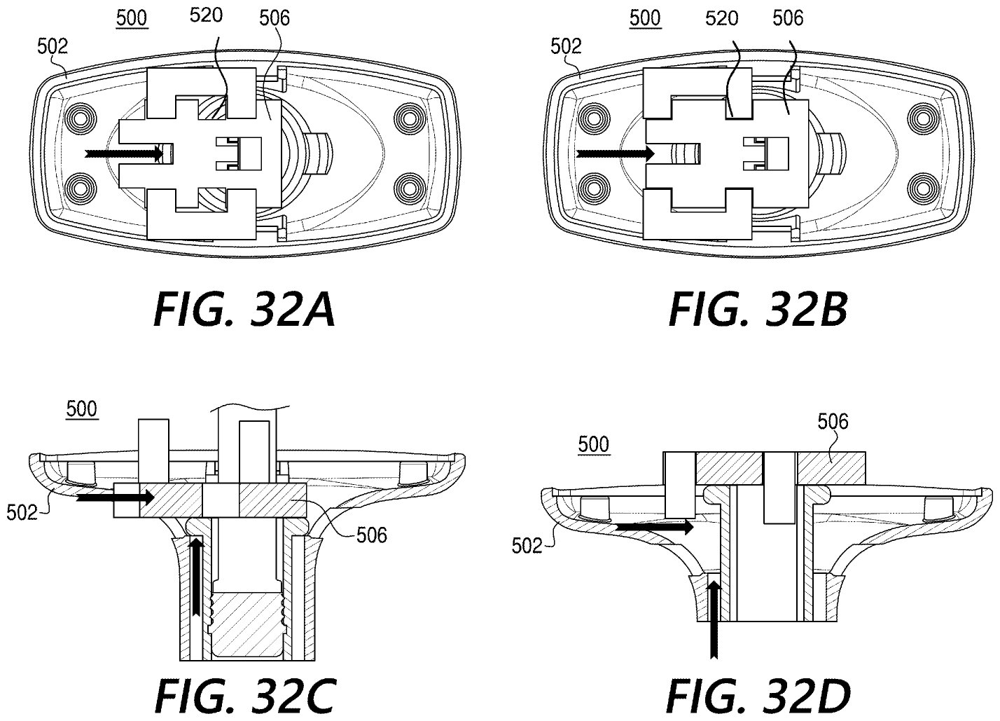

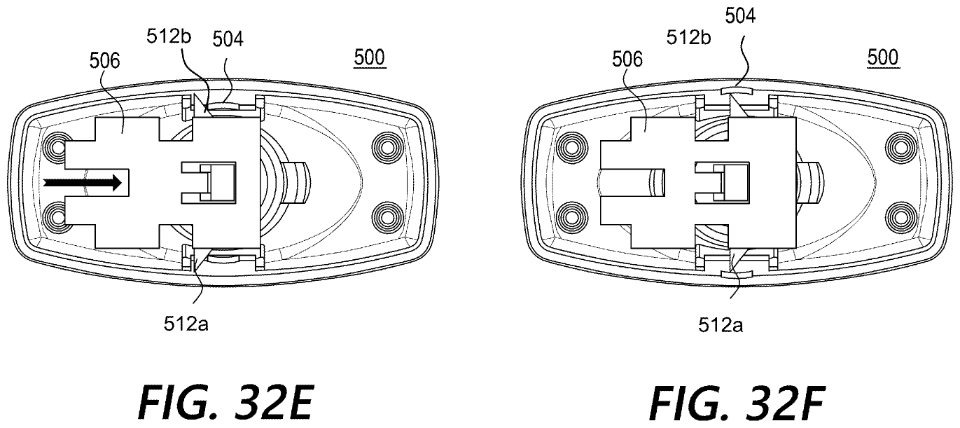

FIGS. 31 and 32A-32F depict another embodiment according to the present disclosure. Features and mechanisms discussed in regards to FIGS. 31 and 32a-32F may be utilized and combined with the features of device 400 as described above and shown in FIGS. 30A-3D. FIGS. 31, 32A, 32B, 32E, and 32F depict a top view of a device 500. Device 500 may include a housing (not shown), finger flange 502, a thumb pad 504, and a slider 506. Thumb pad 504 may include a plurality of clips 508a, 508b. Slider 506 may include an opening 510 for receiving a plunger rod (not shown). Clips 508a, 508b may couple thumb pad 504 and a plunger rod in opening 510. A plunger rod utilized in device 500 and with the features of device 500 discussed here may include various geometric features, e.g, recessed portions as shown in FIG. 19B. Referring to FIGS. 31, 32A, 32B, 32E, and 32F, to initiate injection, a user may press the plunger rod in a distal direction. During injection, as a geometry of the plunger rod changes, e.g., in diameter and/or in width, as described above in regards to device 400 in FIGS. 30A-30D, a spring may push slider 506, e.g., in a right direction. As slider 506 is displaced, ramps 512a, 512b of slider 506 may push against clips 508a, 508b. Ramps 512a, 512b may have a width that is larger than a portion of slider 506 that initially sits between clips 508a, 508b. Ramps 512a, 512b may press against clips 508a, 508b, deflecting clips 508a, 508b in an outward direction. This unlocks thumb pad 504 from the plunger rod, while simultaneously allowing a syringe (not shown) to move in a proximal direction and deflecting clips 508a, 508b in an outward direction to lock into the housing, to a lock-out position. FIG. 31 is a top view of device 500 at end-of-dose. FIGS. 32A-32D depict slider 506 including a recess 520. Recess 520 may be shaped as a C-block, i.e., having a C-shape. Recess 520 may interact with portions of the housing to prevent the slider and/or syringe from moving in a proximal direction. These features may eliminate the need for the user to apply extra force at the end of injection to activate a safety mechanism for lockout out the syringe. A spring 514 may be located on the side of slider 506 including the ramps 512a, 512b. Spring 514 may release its stored energy, pushing the syringe in a proximally direction, simultaneously while thumb pad 504 and the plunger rod decouple from each other.

Components of the devices herein may be made of any suitable material, and each component may be made from the same or different materials as other components. For example, one or more components may be made of a material including a polymer, such as a plastic. In some embodiments, one or more components may include multiple different materials, e.g., glass, plexiglass, any other suitable polymer or copolymer, plastic, or rubber. In some embodiments, a portion of the product container configured to contain a formulated drug substance may be made of a transparent or translucent material. In some embodiments, components of the device may be made of materials each having a similar or different hardness. In some embodiments, components may include elastic materials. For example, components that may be deflectable or flexible, may be made of a material having some flexibility, e.g., to allow for deflection. One or more of the materials listed above (e.g., plastic, rubber, polymers, or copolymers) may have such characteristics.

The description above and examples are illustrative and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description.

Embodiments of the present disclosure may include the following features:

Item 1. A drug delivery device, the device comprising:

a housing;

a product container arranged in the housing;

a plunger rod partially disposed inside the product container; and

a thumb pad configured to receive a portion of the plunger rod,

wherein the thumb pad and the plunger rod are configured to transition from a first state to a second state, wherein at the first state, the plunger rod is prevented from moving in a proximal direction, and at the second state, the plunger rod moves freely in a proximal direction.

Item 2. The device of item 1, wherein the thumb pad comprises an extension projecting distally from the thumb pad.

Item 3. The device of item 2, wherein the housing comprises a shoulder, wherein in the first state, the shoulder projects radially inwards and prevents movement of the product container.

Item 4. The device of item 3, wherein in the second state, the extension of the thumb pad is configured to deflect the shoulder radially outwards to allow the product container to move in a proximal direction.

Item 5. The device of item 4, wherein the product container and a needle attached to a distal end of the product container are retracted into the housing in the proximal direction so that the needle is located proximal to a distal end of the housing.

Item 6. The device of item 5, wherein a distance the product container retracts proximally into the housing in the second state is substantially equal to a length of the needle extending distally from the housing in the first state.

Item 7. The device of item 1, wherein a proximal end portion of the plunger rod includes a flange and the thumb pad includes a recess configured to receive the flange.

Item 8. The device of item 1, wherein the plunger rod abuts an interior distal surface of the thumb pad in the second state.

Item 9. The device of item 1, wherein an audible feedback is produced as a portion of the plunger rod abuts an interior distal surface of the thumb pad in the second state.

Item 10. The device of item 1, wherein the housing includes a surface configured to deflect a portion of the thumb pad in an outward direction to release the proximal portion of the plunger rod when transitioning to the second state.

Item 11. The device of item 1, wherein the housing includes a cutout configured to engage a portion of the thumb pad to couple the thumb pad to the housing in the second state.

Item 12. The device of item 1, wherein the plunger rod includes a first portion and a second portion, the first portion having a diameter greater than a diameter of the second portion, such that in a first state, the first portion is proximal to the second portion and in a second state, the second portion collapses into the first portion.

Item 13. The device of item 1, further including a rotator configured to sit proximal to a proximal end of the product container in a first state and configured to surround the proximal end of the product container in a second state.

Item 14. The device of item 1, wherein the plunger rod includes a first portion and a second portion, a distal end of the second portion being coupled to a proximal end of the first portion, and the housing includes a proximal extension, wherein in a first state, the proximal extension abuts the distal end of the first portion, preventing the product container from moving proximally away from the housing.

Item 15. The device of item 14, wherein in a second state, the proximal extension deflects away from the distal end of the first portion, allowing the product container to move proximally away from housing and into the thumb pad.

Item 16. The device of item 1, wherein a proximal end of the housing includes a rear cap configured to surround a proximal end of the product container.

Item 17. A drug delivery device, the device comprising:

a housing;

a product container disposed in the housing, and a needle extending from a distal end of the product container;

a plunger rod for dispensing a product from the product container, the plunger rod including a first indent at a distal portion of the plunger rod and a second indent at a proximal portion of the plunger rod;

a blocking component at a proximal end of the housing, wherein the plunger rod is slidably received within the blocking component, and wherein the blocking component includes a deflectable arm; and

a needle cover at least partially disposed in the housing.

Item 18. The device of item 17, wherein the device is configured to transition from a first state in which a proximal end of the needle cover abuts the deflectable arm and the deflectable arm is aligned with the first indent, to a second state in which the deflectable arm is deflected radially outward, and to a third state in which the deflectable arm is aligned with the second indent, and the proximal end of the needle cover is located distal to the deflectable arm.

Item 19. The device of item 17, wherein the second indent has a smaller diameter than the first indent.

Item 20. The device of item 17, wherein the housing includes a ledge for abutting a portion of the needle cover to prevent proximal movement of the needle cover into the housing.

Item 21. The device of item 18, wherein, in the first state, the needle cover extends distally of the housing to cover the needle.

Item 22. The device of item 18, wherein, in the second state, the proximal end of the needle cover abuts a distal portion of the blocking component.

Item 23. The device of item 18, wherein, in the first state, the deflectable arm restricts movement of the plunger rod, and wherein in the second state, the deflected deflectable arm permits movement of the plunger rod.

Item 24. The device of item 17, further including an internal rod configured to be secured within the plunger rod.

Item 25. The device of item 24, wherein the internal rod includes a distal block configured to abut a portion of the plunger rod, preventing the plunger rod from moving in a distal direction.

Item 26. The device of item 25, wherein a distal end of the plunger rod includes deflectable arms configured to abut against a portion of the internal rod, preventing the plunger rod from moving in a distal direction.

Item 27. The device of item 25, wherein the plunger rod includes a flexible section, such that in a first state, the distal block prevents the flexible section from compressing and in a second state, the distal block moves away from the flexible section allowing the flexible section to compress.

Item 28. The device of item 17, wherein the housing includes a finger flange and a button mechanism, wherein the button mechanism is configured to abut a portion of the plunger rod in a first state and deflect away from the plunger rod in a second state.

Item 29. A drug delivery device, the device comprising:

a housing;

a plunger rod;

a needle cover disposed at least partially within the housing; and

a rotating component having a track extending along an outer surface of the rotating component,

wherein a portion of the plunger rod extends through a central region of the rotating component, wherein the rotating component is located radially inward of a proximal portion of the needle cover, and wherein a projection of the needle cover engages the track of the rotating component and is configured to move along the track of the rotating component as the device transitions from a first state, to a second state, and to a third state.

Item 30. The device of claim 29, further comprising:

a product container for containing a liquid drug product; and

a stopper disposed within the product container, wherein distal movement of the plunger rod distally moves the stopper, and

wherein, in the first state, the projection engages a first end of the track, and distal movement of the plunger rod is restricted.

Item 31. The device of item 30, wherein, in the second state, the projection engages a middle portion of the track so that distal movement of the plunger rod is permitted, and the needle cover is positioned further proximally within the housing relative to a position of the needle cover in the first state.

Item 32. The device of item 31, wherein, in the third state, the projection engages a second end of the track, and the needle cover is located further distally within the housing relative to a position of the needle cover in the second state to extend from a distal end of the housing.

Item 33. The device of item 32, wherein, in the third state, the needle cover is located further distally within the housing relative to the position of the needle cover in the first state, wherein the needle cover extends from the distal end of the housing in the first state and in the third state.

Item 34. A drug delivery device, the device comprising:

a housing comprising a deflectable tab;

a syringe arranged in the housing;

a plunger rod including a stopper for dispensing a product contained in the syringe;

a needle cover at least partially disposed in the housing, the needle cover including a notch; and

a ring comprising a ledge, the ring positioned around a proximal end of the syringe,

wherein the device is configured to transition between a first state, a second state, and a third state,

wherein in the second state, the needle cover is located further proximally within the housing compared to the first state and the third state, the proximal end of the needle cover is configured to push the deflectable tab of the housing outwards, and the notch of the needle cover engages with the ledge of the ring.

Item 35. The device of item 34, wherein, in the third state, the needle cover and ring are located distal to the deflectable tab, and the deflectable tab restricts movement of the needle cover in the proximal direction.

Item 36. The device of item 35, wherein in the first state, the needle cover is located distal to the ring.

Item 37. A drug delivery device, the device comprising:

a housing;

a product container arranged in the housing; and

a plunger rod partially disposed inside the product container, the plunger rod including a first portion and a second portion, wherein the first portion is configured to receive the second portion,

wherein the first portion and the second portion of the plunger rod are configured to transition from a first state to a second state, wherein at the first state, the second portion of the plunger rod is prevented from moving in a proximal direction and at the second state, the second portion of the plunger rod moves freely in a proximal direction.

Item 38. The device of item 37, wherein the first portion includes a thumb pad.

Item 39. The device of item 38, wherein the thumb pad includes a recess configured to receive the second portion of the plunger rod.

Item 40. The device of item 37, wherein in the second state, the second portion of the plunger rod moves in a proximal direction into the first portion of the plunger rod.

Item 41. The device of item 37, wherein the plunger rod includes a flexible portion.

Item 42. The device of item 37, wherein the second portion of the plunger rod is removably coupled to the first portion of the plunger rod.

What is claimed is:

1. A drug delivery device, the device comprising:
a housing;
a product container arranged in the housing;
a plunger rod partially disposed inside the product container; and
a thumb pad configured to receive a portion of the plunger rod,
wherein the thumb pad and the plunger rod are configured to transition from a first state to a second state, wherein at the first state, the plunger rod is prevented from moving in a proximal direction, and at the second state, the plunger rod moves freely in a proximal direction.

2. The device of claim 1, wherein the thumb pad comprises an extension projecting distally from the thumb pad.

3. The device of claim 2, wherein the housing comprises a shoulder, wherein in the first state, the shoulder projects radially inwards and prevents movement of the product container.

4. The device of claim 3, wherein in the second state, the extension of the thumb pad is configured to deflect the shoulder radially outwards to allow the product container to move in a proximal direction.

5. The device of claim 4, wherein the product container and a needle attached to a distal end of the product container are retracted into the housing in the proximal direction so that the needle is located proximal to a distal end of the housing.

6. The device of claim 5, wherein a distance the product container retracts proximally into the housing in the second state is substantially equal to a length of the needle extending distally from the housing in the first state.

7. The device of claim 1, wherein a proximal end portion of the plunger rod includes a flange and the thumb pad includes a recess configured to receive the flange.

8. The device of claim 1, wherein the plunger rod abuts an interior distal surface of the thumb pad in the second state.

9. The device of claim 1, wherein an audible feedback is produced as a portion of the plunger rod abuts an interior distal surface of the thumb pad in the second state.

10. The device of claim 1, wherein the housing includes a surface configured to deflect a portion of the thumb pad in an outward direction to release the proximal portion of the plunger rod when transitioning to the second state.

11. The device of claim 1, wherein the housing includes a cutout configured to engage a portion of the thumb pad to couple the thumb pad to the housing in the second state.

12. The device of claim 1, wherein the plunger rod includes a first portion and a second portion, the first portion having a diameter greater than a diameter of the second portion, such that in a first state, the first portion is proximal to the second portion and in a second state, the second portion collapses into the first portion.

13. The device of claim 1, further including a rotator configured to sit proximal to a proximal end of the product container in a first state and configured to surround the proximal end of the product container in a second state.

14. The device of claim 1, wherein the plunger rod includes a first portion and a second portion, a distal end of the second portion being coupled to a proximal end of the first portion, and the housing includes a proximal extension, wherein in a first state, the proximal extension abuts the distal end of the first portion, preventing the product container from moving proximally away from the housing.

15. The device of claim 14, wherein in a second state, the proximal extension deflects away from the distal end of the first portion, allowing the product container to move proximally away from housing and into the thumb pad.

16. The device of claim 1, wherein a proximal end of the housing includes a rear cap configured to surround a proximal end of the product container.

17. A drug delivery device, the device comprising:
a housing;
a product container disposed in the housing, and a needle extending from a distal end of the product container;
a plunger rod for dispensing a product from the product container, the plunger rod including a first indent at a distal portion of the plunger rod and a second indent at a proximal portion of the plunger rod;
a blocking component at a proximal end of the housing, wherein the plunger rod is slidably received within the blocking component, and wherein the blocking component includes a deflectable arm; and
a needle cover at least partially disposed in the housing.

18. The device of claim 17, wherein the device is configured to transition from a first state in which a proximal end of the needle cover abuts the deflectable arm and the deflectable arm is aligned with the first indent, to a second state in which the deflectable arm is deflected radially outward, and to a third state in which the deflectable arm is aligned with the second indent, and the proximal end of the needle cover is located distal to the deflectable arm.

19. The device of claim 17, wherein the second indent has a smaller diameter than the first indent.

20. The device of claim 17, wherein the housing includes a ledge for abutting a portion of the needle cover to prevent proximal movement of the needle cover into the housing.

21. The device of claim 18, wherein, in the first state, the needle cover extends distally of the housing to cover the needle.

22. The device of claim 18, wherein, in the second state, the proximal end of the needle cover abuts a distal portion of the blocking component.

23. The device of claim 18, wherein, in the first state, the deflectable arm restricts movement of the plunger rod, and wherein in the second state, the deflected deflectable arm permits movement of the plunger rod.

24. The device of claim 17, further including an internal rod configured to be secured within the plunger rod.

25. The device of claim 24, wherein the internal rod includes a distal block configured to abut a portion of the plunger rod, preventing the plunger rod from moving in a distal direction.

26. The device of claim 25, wherein a distal end of the plunger rod includes deflectable arms configured to abut against a portion of the internal rod, preventing the plunger rod from moving in a distal direction.

27. The device of claim 25, wherein the plunger rod includes a flexible section, such that in a first state, the distal block prevents the flexible section from compressing and in a second state, the distal block moves away from the flexible section allowing the flexible section to compress.

28. The device of claim 17, wherein the housing includes a finger flange and a button mechanism, wherein the button mechanism is configured to abut a portion of the plunger rod in a first state and deflect away from the plunger rod in a second state.

29. A drug delivery device, the device comprising:
a housing;
a plunger rod;
a needle cover disposed at least partially within the housing; and
a rotating component having a track extending along an outer surface of the rotating component,
wherein a portion of the plunger rod extends through a central region of the rotating component, wherein the rotating component is located radially inward of a proximal portion of the needle cover, and wherein a projection of the needle cover engages the track of the rotating component and is configured to move along the track of the rotating component as the device transitions from a first state, to a second state, and to a third state.

30. The device of claim 29, further comprising:
a product container for containing a liquid drug product; and
a stopper disposed within the product container, wherein distal movement of the plunger rod distally moves the stopper, and
wherein, in the first state, the projection engages a first end of the track, and distal movement of the plunger rod is restricted.

31. The device of claim 30, wherein, in the second state, the projection engages a middle portion of the track so that distal movement of the plunger rod is permitted, and the needle cover is positioned further proximally within the housing relative to a position of the needle cover in the first state.

32. The device of claim 31, wherein, in the third state, the projection engages a second end of the track, and the needle cover is located further distally within the housing relative to a position of the needle cover in the second state to extend from a distal end of the housing.

33. The device of claim 32, wherein, in the third state, the needle cover is located further distally within the housing relative to the position of the needle cover in the first state, wherein the needle cover extends from the distal end of the housing in the first state and in the third state.

34. A drug delivery device, the device comprising:
a housing comprising a deflectable tab;
a syringe arranged in the housing;
a plunger rod including a stopper for dispensing a product contained in the syringe;
a needle cover at least partially disposed in the housing, the needle cover including a notch; and
a ring comprising a ledge, the ring positioned around a proximal end of the syringe,
wherein the device is configured to transition between a first state, a second state, and a third state,
wherein in the second state, the needle cover is located further proximally within the housing compared to the first state and the third state, the proximal end of the needle cover is configured to push the deflectable tab of the housing outwards, and the notch of the needle cover engages with the ledge of the ring.

35. The device of claim 34, wherein, in the third state, the needle cover and ring are located distal to the deflectable tab, and the deflectable tab restricts movement of the needle cover in the proximal direction.

36. The device of claim 35, wherein in the first state, the needle cover is located distal to the ring.

37. A drug delivery device, the device comprising:
a housing;
a product container arranged in the housing; and
a plunger rod partially disposed inside the product container, the plunger rod including a first portion and a second portion, wherein the first portion is configured to receive the second portion,
wherein the first portion and the second portion of the plunger rod are configured to transition from a first state to a second state, wherein at the first state, the second portion of the plunger rod is prevented from moving in a proximal direction and at the second state, the second portion of the plunger rod moves freely in a proximal direction.

38. The device of claim 37, wherein the first portion includes a thumb pad.

39. The device of claim 38, wherein the thumb pad includes a recess configured to receive the second portion of the plunger rod.

40. The device of claim 37, wherein in the second state, the second portion of the plunger rod moves in a proximal direction into the first portion of the plunger rod.

41. The device of claim 37, wherein the plunger rod includes a flexible portion.

42. The device of claim 37, wherein the second portion of the plunger rod is removably coupled to the first portion of the plunger rod.
